(19) United States
(12) Patent Application Publication
(10) Pub. No.: US 2026/0114854 A1
(43) Pub. Date: Apr. 30, 2026

(54) ULTRASOUND IMAGING METHOD AND ULTRASOUND IMAGING SYSTEM

(71) Applicant: SHENZHEN MINDRAY BIO-MEDICAL ELECTRONICS CO., LTD., Shenzhen (CN)

(72) Inventors: Yingying SHEN, Shenzhen (CN); Xiaoguo SONG, Shenzhen (CN); Jianxing CAI, Shenzhen (CN); Lei LI, Shenzhen (CN); Shuang WU, Shenzhen (CN)

(73) Assignee: SHENZHEN MINDRAY BIO-MEDICAL ELECTRONICS CO., LTD., Shenzhen (CN)

(21) Appl. No.: 19/372,495

(22) Filed: Oct. 29, 2025

(30) Foreign Application Priority Data
Oct. 29, 2024 (CN) 202411540870.8

(51) Int. Cl. A61B 8/00; A61B 8/08

(52) U.S. Cl. CPC A61B 8/54; A61B 8/085; A61B 8/461; A61B 8/56

(57) ABSTRACT

An ultrasound imaging method and system are provided. Under a full-real-time scanning mode, multiple frames of ultrasound images are sequentially scanned, upon completion of scanning one frame of ultrasound image, the frame is displayed. Under a semi-real-time scanning mode, a scanning action and a first-type action are alternately performed in the scanning period and the pause period respectively, the scanning action includes scanning one frame of ultrasound image in the scanning period or sequentially scanning multiple frames of ultrasound images, and upon completion of scanning one frame of ultrasound image, the frame is displayed; the first-type action includes pausing the scanning of ultrasound images in the pause period, and automatically switching from the pause period to the scanning period when the duration of the pause period reaches.

### CROSS-REFERENCE TO RELATED APPLICATION

This application claims priority to Chinese patent Application No. 202411540870.8 filed on Oct. 29, 2024, the disclosure of which is hereby incorporated by reference in its entirety.

### BACKGROUND

Ultrasound imaging technology enables imaging of numerous organs within the human body to assist doctors in medical diagnosis. This technology utilizes ultrasound waves to scan tissues and organs, wherein images of a target region are acquired by receiving and processing echo signals. Specifically, ultrasound waves are transmitted to a target tissue, the echo signals returned therefrom are received, and ultrasound images of the target tissue are generated based on the received echo signals. Owing to its advantages including non-invasiveness, low cost, and strong real-time performance, ultrasound imaging has become the most extensively applied and frequently utilized diagnostic modality in contemporary medical imaging.

Generally, higher transmit acoustic power in an ultrasound imaging system correlates with improved image quality. However, such power faces dual constraints: physical limitations of the system's components and the need for compliance with regulatory safety standards.

Consequently, methods to enhance the imaging quality of ultrasound imaging systems under these constraints remain a research issue worth exploring.

### SUMMARY

Considering the aforementioned limitations, the present disclosure provides an ultrasound imaging method and an ultrasound imaging system. The technical solutions are detailed in the following embodiments.

The present disclosure relates to the field of ultrasound imaging, and more particularly, to ultrasound imaging methods and ultrasound imaging systems.

According to a first aspect, an ultrasound imaging method, applicable to an ultrasound imaging system, is provided in some embodiments. The ultrasound imaging method may include:
selecting a current imaging mode from a first imaging mode and a second imaging mode;
when the first imaging mode is selected as the current imaging mode, controlling an ultrasound probe to sequentially scan multiple frames of ultrasound images with a first scanning parameter, wherein upon completion of scanning one frame of ultrasound image, said frame is displayed;
when the second imaging mode is selected as the current imaging mode, determining a scanning mode for the second imaging mode, the scanning mode comprising a full-real-time scanning mode and a semi-real-time scanning mode;
when the scanning mode for the second imaging mode is the full-real-time scanning mode, controlling the ultrasound probe to sequentially scan multiple frames of ultrasound images with a second scanning parameter; wherein: upon completion of scanning one frame of ultrasound image, said frame is displayed; and the second scanning parameter and the first scanning parameter are configured such that an average scanning energy required to form one frame of ultrasound image in an imaging region under the second imaging mode is greater than that required under the first imaging mode; and
when the scanning mode for the second imaging mode is the semi-real-time scanning mode, alternately performing a scanning action during a scanning period and a first-type action during a pause period; wherein: the scanning action comprises controlling the ultrasound probe to sequentially scan multiple frames of ultrasound images with a third scanning parameter during the scanning period; upon completion of scanning one frame of ultrasound image, said frame is displayed; the first-type action comprises pausing scanning ultrasound images during the pause period, and switching automatically from the pause period to the scanning period after a duration of the pause period has elapsed; and the third scanning parameter and the first scanning parameter are configured such that an average scanning energy required to form one frame of ultrasound image in an imaging region under the second imaging mode is greater than that required under the first imaging mode.

In some embodiments, the second scanning parameter and the first scanning parameter are further configured such that one or more of the following is satisfied:
a scanning frame rate under the second imaging mode is less than or equal to that under the first imaging mode;
a scanning range under the second imaging mode is less than or equal to that under the first imaging mode;
a scanning density under the second imaging mode is greater than or equal to that under the first imaging mode;
a number of times a same scanning position is scanned in each frame under the second imaging mode is greater than that under the first imaging mode;
a scanning power for each scan frame under the second imaging mode is greater than or equal to that under the first imaging mode;
a scanning power corresponding to a same scanning position in each scan frame under the second imaging mode is greater than that under the first imaging mode;
an average scanning power per unit time under the second imaging mode is greater than or equal to that under the first imaging mode.

In some embodiments, the third scanning parameter and the first scanning parameter are further configured such that one or more of the following is satisfied:
a scanning frame rate during the scanning period in the semi-real-time scanning mode under the second imaging mode is less than or equal to a scanning frame rate under the first imaging mode;
a scanning range under the second imaging mode is less than or equal to that under the first imaging mode;
a scanning density under the second imaging mode is greater than or equal to that under the first imaging mode;
a number of times a same scanning position is scanned in each frame under the second imaging mode is greater than that under the first imaging mode;
a scanning power of each scan frame under the second imaging mode is greater than or equal to that under the first imaging mode;
a scanning power corresponding to a same scanning position in each scan frame under the second imaging mode is greater than that under the first imaging mode;
an average scanning power per unit time under the second imaging mode is greater than or equal to that under the first imaging mode.

In some embodiments, selecting a current imaging mode from a first imaging mode and a second imaging mode comprises one or more of the following:
obtaining a depth of a target region from an ultrasound image, and selecting the second imaging mode as the current imaging mode when the depth of the target region is greater than or equal to a target depth threshold, wherein the target region comprises any one of: a region selected by a user in the ultrasound image, and a lesion region recognized based on the ultrasound image;
calculating a signal intensity obtained by scanning under the first imaging mode, and switching from the first imaging mode to the second imaging mode when the signal intensity is less than a signal intensity threshold;
calculating a signal-to-noise ratio of a signal obtained by scanning under the first imaging mode, and switching from the first imaging mode to the second imaging mode when the signal-to-noise ratio is less than a ratio threshold;
selecting the first imaging mode as the current imaging mode in response to a first instruction input by a user;
selecting the second imaging mode as the current imaging mode in response to a second instruction input by a user.

In some embodiments, the first-type action further comprises: freezing an ultrasound image and/or replaying an ultrasound image during the pause period.

In some embodiments, a time interval between any two frames of ultrasound images scanned under the full-real-time scanning mode is less than the duration of the pause period under the semi-real-time scanning mode.

In some embodiments, the second scanning parameter and the third scanning parameter are configured such that one or more of the following is satisfied:
within the second imaging mode, a scanning frame rate under the full-real-time scanning mode is less than or equal to a scanning frame rate during the scanning period under the semi-real-time scanning mode;
within the second imaging mode, a scanning range under the full-real-time scanning mode is less than or equal to a scanning range during the scanning period under the semi-real-time scanning mode;
within the second imaging mode, a scanning power of each scan frame under the full-real-time scanning mode is less than or equal to a scanning power of each scan frame during the scanning period under the semi-real-time scanning mode;
within the second imaging mode, an average scanning energy required to form one frame of ultrasound image in an imaging region under the full-real-time scanning mode is less than or equal to that under the semi-real-time scanning mode;
within the second imaging mode, an average scanning power per unit time under the full-real-time scanning mode is less than or equal to that under the semi-real-time scanning mode.

In some embodiments, determining a scanning mode for the second imaging mode comprises any one of the following:
obtaining a current scanning item, determining the full-real-time scanning mode as the scanning mode for the second imaging mode when the current scanning item is a first-type scanning item, and determining the semi-real-time scanning mode as the scanning mode for the second imaging mode when the current scanning item is a second-type scanning item;
recognizing a scanned region based on a current ultrasound image, determining the full-real-time scanning mode as the scanning mode for the second imaging mode when a current scanned region is a first-type tissue region, and determining the semi-real-time scanning mode as the scanning mode for the second imaging mode when the current scanned region is a second-type tissue region;
obtaining a number of cross-sectional types to be scanned currently, determining the full-real-time scanning mode as the scanning mode for the second imaging mode when said number is greater than a first number, and determining the semi-real-time scanning mode as the scanning mode for the second imaging mode when said number is less than a second number, wherein the first number is greater than or equal to the second number;
obtaining a current imaging depth, determining the full-real-time scanning mode as the scanning mode for the second imaging mode when the current imaging depth is less than a first depth threshold, and determining the semi-real-time scanning mode as the scanning mode for the second imaging mode when the current imaging depth is greater than a second depth threshold, wherein the second depth threshold is greater than or equal to the first depth threshold;
obtaining a depth of the target region from the ultrasound image, determining the full-real-time scanning mode as the scanning mode for the second imaging mode when the depth of the target region is less than a third depth threshold, and determining the semi-real-time scanning mode as the scanning mode for the second imaging mode when the depth of the target region is greater than a fourth depth threshold; wherein the fourth depth threshold is greater than or equal to the third depth threshold; and the target region includes any one of: a region selected by a user in the ultrasound image, and a lesion region recognized based on the ultrasound image.

In some embodiments, the average scanning energy required to form one frame of ultrasound image is a ratio of a total scanning power required to form one frame of ultrasound image to an area of the imaging region under a corresponding imaging mode, wherein the scanning power comprises an emission power corresponding to a scan point or an emission power corresponding to a scan line.

In some embodiments, the second scanning parameter is configured such that the ultrasound imaging system meets a safety index when working; and/or, the third scanning parameter is configured such that the ultrasound imaging system meets the safety index when working; wherein the safety index comprises at least one of: a mechanical index MI, a temperature, and a thermal index TI.

According to a second aspect, an ultrasound imaging method provided in some embodiments may include:
determining a current scanning mode, the scanning mode comprising a full-real-time scanning mode and a semi-real-time scanning mode;
when the current scanning mode is the full-real-time scanning mode, sequentially scan multiple frames of ultrasound images, wherein after each frame of ultrasound image is scanned, said frame is displayed; and
when the current scanning mode is the semi-real-time scanning mode, alternately performing a scanning action during a scanning period and a first-type action during a pause period, wherein the scanning action comprises scanning one frame of ultrasound image during the scanning period or sequentially scanning multiple frames of ultrasound images; upon completion of scanning one frame of ultrasound image, said frame is displayed; the first-type action comprises pausing the scanning of ultrasound images during the pause period, and switching automatically from the pause period to the scanning period after a duration of the pause period has elapsed.

In some embodiments, the first-type action further comprises: freezing an ultrasound image and/or replaying an ultrasound image during the pause period.

In some embodiments, a time interval between any two frames of ultrasound images scanned under the full-real-time scanning mode is less than the duration of the pause period under the semi-real-time scanning mode.

In some embodiments, a scanning frame rate under the full-real-time scanning mode is less than or equal to a scanning frame rate during the scanning period under the semi-real-time scanning mode.

In some embodiments, a scanning range under the full-real-time scanning mode is less than or equal to a scanning range during the scanning period under the semi-real-time scanning mode.

In some embodiments, a scanning power of each scan frame under the full-real-time scanning mode is less than or equal to a scanning power of each scan frame during the scanning period under the semi-real-time scanning mode.

In some embodiments, an average scanning energy required to form one frame of ultrasound image under the full-real-time scanning mode is less than or equal to that under the semi-real-time scanning mode.

In some embodiments, an average scanning power per unit time under the full-real-time scanning mode is less than or equal to that under the semi-real-time scanning mode.

In some embodiments, determining a current scanning mode comprises any one of the following:
obtaining a current scanning item, determining the full-real-time scanning mode as the current scanning mode when the current scanning item is a first-type scanning item, and determining the semi-real-time scanning mode as the current scanning mode when the current scanning item is a second-type scanning item;
recognizing a scanned region based on a current ultrasound image, determining the full-real-time scanning mode as the current scanning mode when a current scanned region is a first-type tissue region, and determining the semi-real-time scanning mode as the current scanning mode when the current scanned region is a second-type tissue region;
obtaining a number of cross-sectional types to be scanned currently, determining the full-real-time scanning mode as the current scanning mode when said number is greater than a first number, and determining the semi-real-time scanning mode as the current scanning mode when said number is less than or equal to the first number;
obtaining a current imaging depth, determining the full-real-time scanning mode as the current scanning mode when the current imaging depth is less than a first depth threshold, and determining the semi-real-time scanning mode as the current scanning mode when the current imaging depth is greater than a second depth threshold, wherein the second depth threshold is greater than or equal to the first depth threshold;
obtaining a depth of the target region from the ultrasound image, determining the full-real-time scanning mode as the current scanning mode when the depth of the target region is less than a third depth threshold, and determining the semi-real-time scanning mode as the current scanning mode when the depth of the target region is greater than a fourth depth threshold; wherein the fourth depth threshold is greater than or equal to the third depth threshold; and the target region includes any one of: a region selected by a user in the ultrasound image, and a lesion region recognized based on the ultrasound image.

According to a third aspect, an ultrasound imaging method provided in some embodiments may include:
performing a first scan: controlling an ultrasound probe to scan a target tissue to be imaged with a first scanning parameter to obtain a first ultrasound image of the target tissue to be imaged;
displaying the first ultrasound image in a first region of a display interface;
determining a target region based on the first ultrasound image, and determining, in the target tissue to be imaged, a target tissue corresponding to the target region;
performing a second scan: controlling the ultrasound probe to scan the target tissue with a second scanning parameter to obtain a second ultrasound image including the target tissue; and
displaying the second ultrasound image in the first region of the display interface so as to replace the first ultrasound image.

In some embodiments, the second scanning parameter and the first scanning parameter are further configured such that one or more of the following is satisfied:
a scanning density of the target tissue in the second scan is greater than that in the first scan; the scanning density comprises a point density or a line density;
a number of times a same scanning position is scanned in each frame of the target tissue in the second scan is greater than that in the first scan; the scanning position comprises a scan point or a scan line;
a scanning power of each scan frame of the target tissue in the second scan is greater than that in the first scan;
a scanning power corresponding to a same scanning position of the target tissue in each scan frame in the second scan is greater than that in the first scan;
an average scanning energy required to form one frame of ultrasound image in the second scan is greater than or equal to that in the first scan.

In some embodiments, determining a target region based on the first ultrasound image comprises any one of the following:
receiving a region selection operation by a user on the first ultrasound image, determining a region corresponding to the region selection operation on the first ultrasound image, and taking it as the target region;
performing recognition on the first ultrasound image to obtain a recognition result, and determining the target region on the first ultrasound image based on the recognition result.

In some embodiments, the recognition result comprises a target anatomical structure or a lesion structure.

According to a fourth aspect, an ultrasound imaging method, applicable to an ultrasound imaging system, is provided in some embodiments. The ultrasound imaging method may include:
selecting a current imaging mode from a conventional imaging mode and a high-penetration imaging mode;
when the conventional imaging mode is selecting as the current imaging mode, controlling an ultrasound probe to sequentially scan multiple frames of ultrasound images with a first scanning parameter, wherein upon completion of scanning one frame of ultrasound image, said frame is displayed;
when the high-penetration imaging mode is selecting as the current imaging mode, controlling an ultrasound probe to sequentially scan multiple frames of ultrasound images with a second scanning parameter, wherein upon completion of scanning one frame of ultrasound image, said frame is displayed;
the second scanning parameter and the first scanning parameter are configured such that any combination of the following is satisfied:
a scanning density within a target scanning range under the high-penetration imaging mode is higher than that under the conventional imaging mode;
a scanning density within a non-target scanning range under the high-penetration imaging mode is lower than that under the conventional imaging mode;
a number of times a same scanning position is scanned in each frame within the target scanning range under the high-penetration imaging mode is greater than that under the conventional imaging mode;
a number of times a same scanning position is scanned in each frame within the non-target scanning range under the high-penetration imaging mode is lower than that under the conventional imaging mode;
a scanning range under the high-penetration imaging mode is smaller than that under the conventional imaging mode;
a scanning frame rate under the high-penetration imaging mode is lower than that under the conventional imaging mode;
a transmission frequency under the high-penetration imaging mode is lower than that under the conventional imaging mode;
a scanning power corresponding to a same scanning position in each scan frame under the high-penetration imaging mode is higher than that under the conventional imaging mode.

In some embodiments, selecting a current imaging mode from a conventional imaging mode and a high-penetration imaging mode comprises:
obtaining a depth of a target region from an ultrasound image, and when the depth of the target region is greater than or equal to a target depth threshold, selecting the high-penetration imaging mode as the current imaging mode; wherein the target region comprises any one of: a region selected by a user in the ultrasound image, and a lesion region recognized based on the ultrasound image;
calculating a signal intensity obtained by scanning under the conventional imaging mode, and when the signal intensity is less than a signal intensity threshold, switching from the conventional imaging mode to the high-penetration imaging mode; and
calculating a signal-to-noise ratio of a signal obtained by scanning under the conventional imaging mode, and when the signal-to-noise ratio is less than a ratio threshold, switching from the conventional imaging mode to the high-penetration imaging mode.

In some embodiments, the second scanning parameter is configured such that the ultrasound imaging system meets a safety index when working; the safety index includes at least one of: a mechanical index MI, a temperature, and a thermal index TI.

According to a fifth aspect, an ultrasound imaging method, applicable to an ultrasound imaging system, is provided in some embodiments. The ultrasound imaging method may include:
selecting a current imaging mode from a first imaging mode and a second imaging mode;
when the first imaging mode is selecting as the current imaging mode, controlling an ultrasound probe to sequentially scan multiple frames of ultrasound images with a first scanning parameter, wherein upon completion of scanning one frame of ultrasound image, said frame is displayed;
when the second imaging mode is selecting as the current imaging mode, determining a scanning mode for the second imaging mode, the scanning mode including a full-real-time scanning mode and a semi-real-time scanning mode;
when the scanning mode for the second imaging mode is the full-real-time scanning mode, controlling an ultrasound probe to sequentially scan multiple frames of ultrasound images with a second scanning parameter, wherein upon completion of scanning one frame of ultrasound image, said frame is displayed; and
when the scanning mode for the second imaging mode is the semi-real-time scanning mode, alternately performing a scanning action during a scanning period and a first-type action during a pause period, wherein the scanning action comprises controlling the ultrasound probe to sequentially scan multiple frames of ultrasound images with a third scanning parameter during the scanning period; upon completion of scanning one frame of ultrasound image, said frame is displayed; the first-type action comprises pausing the scanning of ultrasound images during the pause period, and switching automatically from the pause period to the scanning period after a duration of the pause period has elapsed.

In some embodiments, the second scanning parameter and the first scanning parameter are configured such that an average scanning energy required to form one frame of ultrasound image under the second imaging mode is greater than that required under the first imaging mode for at least one frame. In some embodiments, the third scanning parameter and the first scanning parameter are configured such that an average scanning energy required to form one frame of ultrasound image under the second imaging mode is greater than that required under the first imaging mode for at least one frame.

According to a sixth aspect, an ultrasound imaging system provided in some embodiments may include an ultrasound probe, a transmit and receive control circuit, a processor, and a display; wherein:
the ultrasound probe is configured to transmit ultrasound waves to a target object and receive echo signals thereof;
the transmit and receive control circuit is configured to control the ultrasound probe to transmit ultrasound waves and receive the echo signals thereof;
the processor is configured to process the echo signals to generate ultrasound images;
the display is configured to display the ultrasound images; and
the processor is further configured to execute the method according to any one of embodiments disclosed herein.

According to the ultrasound imaging method and ultrasound imaging system of the above embodiments, two scanning modes, namely the full-real-time scanning mode and the semi-real-time scanning mode, are proposed. Different scanning modes may be selected according to specific scenarios to meet the imaging quality requirements.

According to the ultrasound imaging method and ultrasound imaging system of the above embodiments, by determining a target region and a target tissue from the first ultrasound image and reducing the scanning region to the target tissue corresponding to the target region, the imaging quality of the target region can be improved while ensuring that the ultrasound imaging system meets the safety index during operation.

According to the ultrasound imaging method and ultrasound imaging system of the above embodiments, the conventional imaging mode and the high-penetration imaging mode are proposed. Different imaging modes may be selected according to specific scenarios, to meet the imaging quality requirements while ensuring that the ultrasound imaging system 100 meets the safety index during operation.

### BRIEF DESCRIPTION OF THE DRAWINGS

FIG. 1 is a structural schematic diagram of an ultrasound imaging system in some embodiments;

FIG. 2 is a flowchart of an ultrasound imaging method in some embodiments;

FIG. 3 is a schematic diagram comparing the imaging region and scan lines between the first imaging mode and the second imaging mode in some embodiments;

FIG. 4A is a schematic diagram comparing scanning under the first imaging mode and the second imaging mode with the latter operating in the full-real-time scanning mode in some embodiments;

FIG. 4B is another schematic diagram comparing scanning under the first imaging mode and the second imaging mode with the latter operating in the full-real-time scanning mode in some embodiments;

FIG. 5 is a schematic diagram comparing scanning under the first imaging mode and the second imaging mode with the latter operating in the full-real-time scanning mode in some embodiments;

FIG. 6A is a schematic diagram comparing scanning under the first imaging mode and the second imaging mode with the latter operating in the semi-real-time scanning mode in some embodiments;

FIG. 6B is a schematic diagram comparing scanning under the first imaging mode and the second imaging mode with the latter operating in the semi-real-time scanning mode in some embodiments;

FIG. 7 is a schematic diagram comparing scanning under the first imaging mode and the second imaging mode with the latter operating in the semi-real-time scanning mode in some embodiments;

FIG. 8 is a flowchart of an ultrasound imaging method in some embodiments;

FIG. 9 is a flowchart of an ultrasound imaging method in some embodiments; and

FIG. 10 is a flowchart of an ultrasound imaging method in some embodiments.

### DETAILED DESCRIPTION

Specific embodiments of the present disclosure will now be described in detail with reference to the accompanying drawings. Similar or related components in different embodiments are labeled with associated reference numerals. The following embodiments include detailed descriptions to facilitate understanding of the present disclosure. However, those skilled in the art will readily recognize that certain features may be omitted under specific circumstances or substituted by other components, materials, or methods. In some instances, certain operations related to the present disclosure are not explicitly described or illustrated herein. This intentional exclusion is intentional to avoid obscuring the core technical solutions of the present disclosure. For those skilled in the art, a complete understanding of these operations can be attained through the descriptions provided in this specification and general technical knowledge in the art.

Additionally, the features, operations, or characteristics described in the specification may be combined in any suitable manner to form various embodiments. Similarly, steps or actions in the method descriptions may be reordered or modified in ways that would be obvious to those skilled in the art. Therefore, the sequences presented in the specification and drawings are intended solely to clarify the description of specific embodiments and do not imply mandatory orderings, unless explicitly stated that a particular sequence is required.

The numerical designations assigned to components in this specification, such as ‘first,’ ‘second,’ or similar ordinal terms, serve solely to distinguish described objects and carry no inherent sequential or technical implications. Furthermore, the terms ‘connected’ and ‘coupled’ as used herein encompass both direct and indirect connection (coupling), unless explicitly stated otherwise.

Referring to FIG. 1, some embodiments provide an ultrasound imaging system 100, which includes an ultrasound probe 10, a transmit and receive control circuit 20, a processor 30, and a display 40, as described in detail below.

The ultrasound probe 10 is configured to emit ultrasound waves to a target object (such as the tissue of a human or animal body) and receive the echo signals of the ultrasound waves. In some embodiments, the ultrasound probe 10 includes a plurality of transducers, which are configured to convert between electrical pulse signals and ultrasound waves, thereby enabling the emission of ultrasound waves to the target object and the reception of ultrasound echoes reflected by the tissue to obtain the echo signals of the ultrasound waves. In some embodiments, the plurality of transducers included in the ultrasound probe 10 may be arranged in a row to form a linear array. In some embodiments, the plurality of transducers included in the ultrasound probe 10 may be arranged in a two-dimensional matrix to form a planar array. The transducers, for example, may be piezoelectric crystals, which convert electrical signals into ultrasound signals according to the transmit sequence transmitted by the transmit and receive control circuit 20. Depending on the application, the emitted ultrasound waves (ultrasound signals) may include one or more scanning pulses, one or more reference pulses, one or more push pulses, and/or one or more Doppler pulses. According to the wave morphology, the ultrasound signals include focused waves, plane waves and divergent waves. The transducers are configured to emit ultrasound waves according to the excitation electrical signals or convert the received ultrasound waves into electrical signals. Therefore, each transducer can be used to achieve the mutual conversion between electrical pulse signals and ultrasound waves, thereby enabling the emission of ultrasound waves to the target object and also being used to receive the echo signals of the ultrasound waves reflected by the tissue. During an ultrasound examination, the transmit and receive control circuit 20 can control which transducers are used to emit ultrasound wave beams (emitting transducers) and which transducers are used to receive ultrasound wave beams (receiving transducers), or control the transducers to emit ultrasound waves or receive their echoes in time slots. The transducers involved in the emission of ultrasound waves may be simultaneously excited by electrical signals to simultaneously emit ultrasound waves; or the transducers involved in the emission of ultrasound waves may also be excited by several electrical signals with a certain time interval, thereby emitting ultrasound waves continuously at certain time intervals.

In some examples, the target object may be selected by users. For example, when the ultrasound image is displayed on the display 40, the region of interest on the ultrasound image may be selected by a user to determine the target tissue to be scanned. In some examples, the processor 30 may automatically determine the position of the region of interest on the ultrasound image based on relevant machine recognition algorithms to determine the target tissue to be scanned. In some examples, the target object may also be obtained through semi-automatic detection, so as to determine the target tissue to be scanned. For example, the processor 30 may first automatically detect the position of the target object on the basic ultrasound image based on machine recognition algorithms, and then a user may further modify or correct it to obtain a more accurate position of the target object.

The user may move the ultrasound probe 10 to select an appropriate position and angle, so that the ultrasound probe emits ultrasound waves to the target object, receives the ultrasound echoes returned by the target object, and outputs ultrasound echo signals. The ultrasound echo signals are channel analog electrical signals formed with the receiving transducers as channels, which can carry information such as amplitude information, frequency information, and/or time information.

The transmit and receive control circuit 20 is configured to control the ultrasound probe 10 to emit ultrasound waves and receive their echo signals. For example, the transmit and receive control circuit 20 controls the ultrasound probe 10 to emit ultrasound waves to the target object, and also controls it to receive ultrasound echoes reflected by the tissue. In some embodiments, the transmit and receive control circuit 20 is configured to generate the transmit sequence and the receive sequence, and output them to the ultrasound probe 10. The transmit sequence is configured to control some or all of the plurality of transducer elements in the ultrasound probe 10 to emit ultrasound waves towards the target object. Parameters of the transmit sequence include the number of transducer elements used for transmission and ultrasound transmission parameters (e.g., amplitude, frequency, number of transmissions, emission interval, emission angle, waveform, and/or focusing position, etc.). The receive sequence is configured to control some or all of the plurality of transducer elements to receive the echoes of the ultrasound waves that have passed through the tissue. Parameters of the receive sequence include the number of transducer elements used for reception and echo reception parameters (e.g., reception angle, depth, etc.). the ultrasound wave parameters in the transmit sequence and the echo parameters in the receive sequence vary depending on the intended uses of the ultrasound echoes or the type of images generated generated from the ultrasound echoes. For example, under different working modes, such as B mode, C mode, M mode and D mode (Doppler mode), the transmit sequence parameters may be different. After the echo signals are received by the ultrasound probe 10 under the transmit and receive control circuit 20, and processed by subsequent modules and corresponding algorithms, they can generate B-mode images reflecting tissue anatomy, C-mode images reflecting blood flow information, and D-mode images reflecting Doppler spectrum images, etc. It should be understood that the ultrasound images in the present disclosure may be two-dimensional images (e.g., the aforementioned B-mode images), or one-dimensional images (e.g., the aforementioned D-mode images).

The processor 30 is configured to process the ultrasound echo signals received by the ultrasound probe 10 (i.e., the echo signals of the ultrasound waves), and may perform data processing in one or more steps, such as receiving and forming channel data, analog-to-digital conversion, signal demodulation, amplification, filtering, downsampling, beamforming, magnitude computation, logarithmic compression, and grayscale transformation, etc. Each data processing step is described below.

The transducer elements of the ultrasound probe 10 receive ultrasound echo signals and convert them into electrical signal (analog data), which are then converted into digital signals through analog-to-digital conversion, referred to as channel acquisition data. The signal demodulation step involves demodulating the input ultrasound data, where the input ultrasound data may be the digital signal obtained after analog-to-digital conversion. Demodulation methods may include: simple demodulation, quadrature demodulation, Hilbert transform demodulation, secondary sampling demodulation, multiple sampling demodulation or baseband sampling demodulation, etc. The most commonly used demodulation method is quadrature demodulation. That is, the received echo signals are split into two paths, which are multiplied by cos (ωnTₛ) and sin (ωnTₛ) respectively. The amplification processing step includes: amplifying the ultrasound data with different amplification factors according to their reception times to compensate for signal attenuation; or, amplifying the ultrasound data with different amplification factors according to their positions to compensate for signal attenuation. The amplification processing step may be performed after the signal demodulation step.

The filtering stage is typically performed after signal demodulation; for example, a low-pass filter is used to improve signal quality. Downsampling serves to reduce the sampling rate of the signal and lower the computational load. Data normalization can include scaling normalization or standard normalization, and serves to confine data to a specific range, thereby eliminating adverse effects caused by abnormal sample data.

Principal component analysis involves: centering the ultrasound data to obtain centered features, calculating the covariance matrix of these features, solving for the eigenvalues of the covariance matrix, selecting the eigenvectors corresponding to the largest eigenvalues, and projecting the ultrasound data onto these eigenvectors. Principal component analysis primarily functions to reduce the dimensionality of data features.

Data augmentation involves translating and/or adding noise to ultrasound data, and serves to improve the neural network's data processing accuracy. For example, during neural network training, with limited training data, operations such as translation and noise addition are performed to expand the dataset size, thereby improving the neural network's accuracy.

Data rearrangement involves reordering ultrasound data using at least one of the following methods: arranging the demodulated ultrasound data received by each transducer element of the ultrasound probe 10 into two columns (one for I data and one for Q data); for a given transducer element with received data of dimension (Npoint*1), the data may be arranged into two columns (after demodulation) or one column (before demodulation); arranging the ultrasound echo data received by all active transducer elements of the ultrasound probe 10 following a single ultrasound transmission into a matrix (where n is the number of active transducer elements, and the data dimension is (Npoint*2 n)); and dividing the ultrasound echo data received by each element of the ultrasound probe 10 (dimension (Npoint*1)) into multiple segments (e.g., m segments) and arranging them into a matrix. It should be noted that pre-demodulation data is not arranged into two columns. Additionally, in other examples, the data may be combined into 3D or even higher-dimensional data as input. The rearranged data is fed into the neural network as input data, which can improve the network's accuracy.

Beamforming refers to the process of reconstructing channel echo data (which may be RF signals prior to demodulation or baseband signals after demodulation) from the channel domain (e.g., data dimensions: time direction*number of channels*number of transmissions) into beam domain data (i.e., beamforming data; e.g., data dimensions: number of longitudinal points*number of transverse points, corresponding to actual physical spatial points). Beamforming can be implemented using various methods, including but not limited to Delay-and-Sum (DAS), adaptive beamforming, and coherent factor beamforming.

Magnitude computation, logarithmic compression, and grayscale transformation are processing steps performed on ultrasound data within the image domain. These three steps may also be collectively termed scan conversion.

The processor 30 processes ultrasound echo signals and ultimately generates ultrasound images for display on the display 40.

In some embodiments, the processor 30 includes, but is not limited to, a Central Processing Unit (CPU), a Microcontroller Unit (MCU), a Field-Programmable Gate Array (FPGA), a Digital Signal Processor (DSP), and other devices configured to interpret computer instructions and process data in computer software.

In some embodiments, the processor 30 is configured to execute computer applications stored in the non-transitory computer-readable storage medium, thereby performing corresponding steps of the methods. For example, the processor 30 may be implemented using software, hardware, firmware, or a combination thereof, and may employ circuits, one or more Application Specific Integrated Circuits (ASICs), Digital Signal Processors (DSPs), Digital Signal Processing Devices (DSPDs), Programmable Logic Devices (PLDs), Field Programmable Gate Arrays (FPGAs), Central Processing Units (CPUs), controllers, microcontrollers, microprocessors, or any combination thereof, enabling it to perform some, all, or any combination of the steps of the ultrasound imaging methods in the embodiments of this application.

The display 40 is configured to display information, such as parameters and/or images generated by the processor 30; this will be further explained below.

The ultrasound echo signal is closely related to the transmission power and exhibits a strong positive coupling relationship. Meanwhile, the transmission power and other transmission parameters of the ultrasound imaging system 100 also determine the safety of the acoustic output. For example, in accordance with relevant industry requirements, acoustic output safety indicators include the mechanical index (MI) and thermal index (TI) (which characterize potential biological effects), the spatial peak temporal average acoustic intensity (which characterizes acoustic exposure), and the probe surface temperature. Therefore, during operation, the ultrasound imaging system 100 is restricted by the aforementioned acoustic output safety indicators, with these constraints reflected in all aspects of ultrasound scanning; their ultimate manifestation is that the transmission power of the ultrasound imaging system 100 is limited. In addition, the transmission power is also limited by the maximum transmission capability of the ultrasound imaging system 100.

This application provides solutions to these problems, enabling improved imaging quality of the ultrasound imaging system 100, particularly when the system complies with safety indicators in operation.

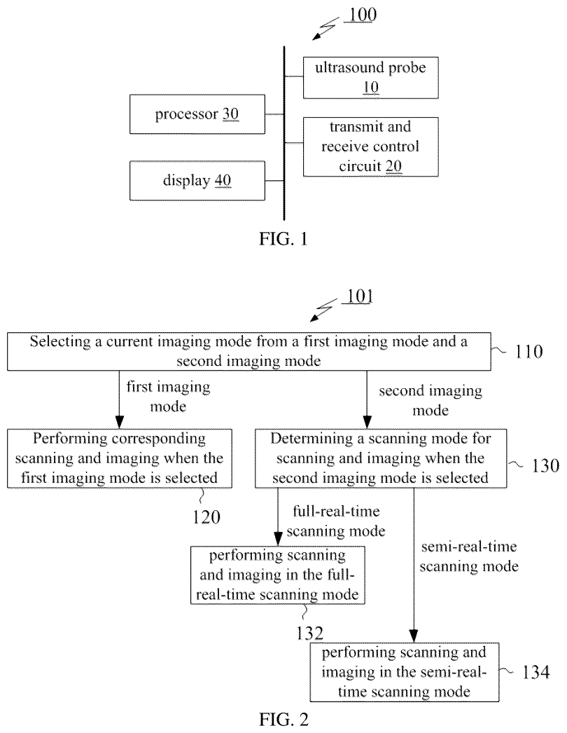

Referring to FIG. 2, some embodiments disclose an ultrasound imaging method 101, which can be applied to the ultrasound imaging system 100 of any embodiment of the present disclosure. In some embodiments, the ultrasound imaging method 101 includes the following steps:

Step 110: Selecting a current imaging mode from a first imaging mode and a second imaging mode.

The ultrasound imaging system 100 may automatically select the current imaging mode from the first and second imaging mode, or may manually select the current imaging mode from the first imaging mode and the second imaging mode based on user input.

In some embodiments, step 110 involves obtaining the depth of a target region from the ultrasound image, and selecting the second imaging mode as the current imaging mode when the depth of the target region is greater than or equal to a target depth threshold. In some examples, the target region includes one of the following: a region selected by a user in the ultrasound image, and a lesion region recognized from the ultrasound image.

In some embodiments, step 110 involves calculating a signal intensity obtained by scanning under the first imaging mode, and switching from the first imaging mode to the second imaging mode when the signal intensity is less than a signal intensity threshold.

In some embodiments, step 110 involves calculating the signal-to-noise ratio of a signal obtained by scanning under the first imaging mode, and switching from the first imaging mode to the second imaging mode when the signal-to-noise ratio is less than a ratio threshold.

In some embodiments, step 110 involves: in response to a first instruction input by a user, selecting the first imaging mode as the current imaging mode. In some embodiments, step 110 involves: in response to a second instruction input by a user, selecting the second imaging mode as the current imaging mode.

For example, a first control corresponding to the first imaging mode and a second control corresponding to the second imaging mode can be provided on the display interface of the display 40. When the first control is operated by a user, the first instruction can be generated; and when the second control is operated, the second instruction can be generated.

Step 120: Performing corresponding scanning and imaging when the first imaging mode is selected.

In some embodiments, step 120 involves controlling the ultrasound probe 10 with a first scanning parameter to sequentially scan multiple frames of ultrasound images when the first imaging mode is selected as the current imaging mode; wherein upon completion of scanning one frame of ultrasound image, said frame is displayed. As mentioned above, the ultrasound image may be two-dimensional or one-dimensional. Thus, when reference is made herein to displaying a frame of ultrasound image, it may be a B-mode or C-mode image displayed two-dimensionally, or a PW image displayed in a line style (i.e., one-dimensionally, sequentially).

In some embodiments, the first scanning parameter is configured such that the ultrasound imaging system 100 meets a safety index when working. The safety index may include at least a mechanical index MI, a temperature, or a thermal index TI, etc.

Step 130: Determining a scanning mode for scanning and imaging when the second imaging mode is selected.

In some embodiments, step 130 involves, when the second imaging mode is selected as the current imaging mode, determining the scanning mode for the second imaging mode. The scanning mode includes a full-real-time scanning mode and a semi-real-time scanning mode; that is, the scanning mode for the second imaging mode needs to be determined from these two modes.

The ultrasound imaging system 100 can automatically determine the scanning mode for the second imaging mode, or can manually select the scanning mode for the second imaging mode based on user operation.

In some embodiments, step 130 involves acquiring a current scanning item, determining the full-real-time scanning mode as the scanning mode for the second imaging mode when the current scanning item is a first-type scanning item, and determining the semi-real-time scanning mode as the scanning mode for the second imaging mode when the current scanning item is a second-type scanning item.

Scanning items are generally defined for a scanned region, such as heart scan, liver scan, abdominal scan, kidney scan, thyroid scan, etc. Scanning items can be pre-classified into the first-type scanning items and the second-type scanning items. The classification may be based on the number of slice types to be scanned in the scanning protocol corresponding to a scanning item: if the number is large (e.g., greater than a first number), it is categorized as the first-type scanning item; and if the number is small (e.g., less than a second number), it is categorized as the second-type scanning item, where the first number is greater than or equal to the second number. Another basis for classification may be the depth of the scanned region from the scanning body surface: if the depth is less than a first depth, it is categorized as the first-type scanning item, and if the depth is greater than the second depth, it is categorized as the second-type scanning item, where the second depth is greater than or equal to the first depth.

In some embodiments, step 130 involves recognizing the scanned region based on the current ultrasound image, determining the full-real-time scanning mode as the scanning mode for the second imaging mode when the current scanned region is a first-type tissue region, and determining the semi-real-time scanning mode as the scanning mode for the second imaging mode when the current scanned region is a second-type tissue region. Step 130 may involve recognizing the scanned region based on the ultrasound image using traditional image recognition or machine learning.

In some embodiments, step 130 involves acquiring the number of currently pending slice types to be scanned, determining the full-real-time scanning mode as the scanning mode for the second imaging mode when the number is greater than the first number, and determining the semi-real-time scanning mode as the scanning mode for the second imaging mode when the number is less than the second number; wherein the first number is greater than or equal to the second number. If, during a single scanning process, the focus is on overall changes and multiple slices of the same region or different regions need to be checked, the full-real-time scanning mode may be selected as the current scanning mode. Conversely, if the focus is more on details of different regions within the same slice, the semi-real-time scanning mode may be selected as the current scanning mode.

In some embodiments, step 130 involves acquiring a current imaging depth, determining the full-real-time scanning mode as the scanning mode for the second imaging mode when the current imaging depth is less than a first depth threshold, and determining the semi-real-time scanning mode as the scanning mode for the second imaging mode when the current imaging depth is greater than a second depth threshold. In some embodiments, the second depth threshold is greater than or equal to the first depth threshold.

In some embodiments, step 130 involves acquiring the depth of a target region from the ultrasound image, determining the full-real-time scanning mode as the scanning mode for the second imaging mode when the depth of the target region is less than a third depth threshold, and determining the semi-real-time scanning mode as the scanning mode for the second imaging mode when the depth of the target region is greater than a fourth depth threshold; wherein the fourth depth threshold is greater than or equal to the third depth threshold. In some examples, the target region includes one of the following: a region selected by a user in the ultrasound image, and a lesion region recognized from the ultrasound image.

When the scanning mode for the second imaging mode is the full-real-time scanning mode, step 132 is executed: performing scanning and imaging in this mode.

In some embodiments, step 132 involves controlling the ultrasound probe 10 to sequentially scan multiple frames of ultrasound images with the second scanning parameter; wherein, upon completion of scanning one frame of ultrasound image, said frame is displayed.

In some embodiments, the second scanning parameter and the first scanning parameter are configured such that an average scanning energy required to form one frame of ultrasound image under the second imaging mode is greater than that required under the first imaging mode for at least one frame.

In some embodiments, the second scanning parameter and the first scanning parameter are configured such that the average scanning energy required to form one frame of ultrasound image under the second imaging mode is greater than the average scanning energy required to form one frame of ultrasound image under the first imaging mode.

In some examples, the average scanning energy required to form one frame of ultrasound image is the ratio of a total scanning power required to form one frame of ultrasound image to the area of an imaging region under the corresponding imaging mode, where the scanning power includes an emission power corresponding to a scan point or an emission power corresponding to a scan line. That is, the average scanning energy required to form one frame of ultrasound image refers to the ratio of total scanning power of this frame of ultrasound image to the area of the imaging region of this frame of ultrasound image, thus it characterizes the scanning power per unit area of the imaging region during the imaging process. Generally, the imaging region is also the scanning region or scanning range.

FIG. 3 is an example in which the box represents the imaging region, and the solid lines inside the box represent the scan lines; the imaging regions of the first imaging mode and the second imaging mode are the same, which is reflected in the fact that the image regions of their respective ultrasound images are identical; the imaging region under the first imaging mode requires scanning of 8 scan lines when generating one frame of ultrasound image, and the imaging region under the second imaging mode also requires scanning of these 8 scan lines when generating one frame of ultrasound image. However, the scanning power of each scan line under the second imaging mode (i.e., the emission power of the ultrasound probe 10 corresponding to the scan line) is greater than that of the corresponding scan line under the first imaging mode, thereby resulting in the scanning power per unit area of the imaging region under the second imaging mode being greater than that under the first imaging mode. That is, the average scanning energy required to form one frame of ultrasound image under the second imaging mode is greater than that under the first imaging mode.

In some embodiments, the second scanning parameter and the first scanning parameter are further configured such that one or more of the following is satisfied:
(1) The scanning frame rate of the second imaging mode is less than or equal to the scanning frame rate of the first imaging mode;
(2) The scanning range of the second imaging mode is less than or equal to the scanning range of the first imaging mode;
(3) The scanning density of the second imaging mode is greater than or equal to the scanning density of the first imaging mode;
(4) The number of times the same scanning position is scanned in each frame under the second imaging mode is greater than that in each frame under the first imaging mode;
(5) The scanning power of each scan frame under the second imaging mode is greater than or equal to that under the first imaging mode;
(6) The scanning power corresponding to the same scanning position in each scan frame under the second imaging mode is greater than that under the first imaging mode;
(7) The average scanning power per unit time under the second imaging mode is greater than or equal to that under the first imaging mode.

The concept of “each scan frame” or “one scan frame” mentioned herein refers to a complete scan of a target range, which is defined as one scan frame. Generally, the ultrasound data (ultrasound echo signals) obtained from one scan frame can be processed to generate one ultrasound image. However, in some cases, multiple sets of ultrasound data (ultrasound echo signals) from multiple scan frames are combined to synthesize one ultrasound image. In this specification, unless specified as a scan frame, the term “one frame” or “each frame” mentioned elsewhere refers to one ultrasound image.

Therefore, the time affecting the scanning frame rate includes two parts: one is the time it takes for a scan frame to scan from start to finish, that is, the scanning time of the scan frame itself; the other is the time interval between adjacent scan frames, that is, the interval from the end of scanning of the previous scan frame to the start of scanning of the next scan frame.

For item (4) above, the number of times the same scanning position is scanned in each ultrasound image of the second imaging mode is greater than that in each ultrasound image of the first imaging mode. Further, their scanning range may also be the same, and the scanning density may also be the same. The scanning position can be a scan point or a scan line. Taking the scan line as an example, if scanning one ultrasound image requires 8 scan lines to be scanned from left to right: under the first imaging mode, a single left-to-right scan is performed, with each scan line scanned once to generate one ultrasound image; under the second imaging mode, multiple left-to-right scans can be performed (so each scan line is scanned multiple times), or scanning can start with the first scan line, each line is scanned multiple times before moving to the next (which is also scanned multiple times), until the last scan line is completed, and then the data is synthesized into one ultrasound image.

For item (6) above, the scanning power corresponding to the same scanning position in each scan frame under the second imaging mode is greater than that in each scan frame under the first imaging mode. Further, their scanning range can be the same, and their scanning density may also be the same. The scanning position can be a scan point or a scan line. The example in FIG. 3 above serves as an illustration and will not be repeated here.

The above situations can be adaptively combined based on their mutual relationships.

For example, the second scanning parameter and the first scanning parameter are configured such that: the scanning frame rate of the second imaging mode is less than that of the first imaging mode, and the scanning range of the second imaging mode is less than or equal to that of the first imaging mode; furthermore, the scanning power of each scan frame under the second imaging mode is greater than that under the first imaging mode, for example, their scanning densities may be the same, but the scanning power corresponding to the same scanning position in each scan frame under the second imaging mode is greater than that under the first imaging mode; additionally, the time taken for each scan frame under the second imaging mode is greater than that under the first imaging mode, and the time interval between adjacent scan frames under the second imaging mode is greater than that under the first imaging mode; or, the time taken for each scan frame under the second imaging mode is greater than that under the first imaging mode, and the time interval between adjacent scan frames under the second imaging mode is equal to that under the first imaging mode; or, the time taken for each scan frame under the second imaging mode is equal to that under the first imaging mode, and the time interval between adjacent scan frames under the second imaging mode is greater than that under the first imaging mode.

For example, the second scanning parameter and the first scanning parameter are configured such that: the scanning frame rate of the second imaging mode is less than that of the first imaging mode, and the scanning range of the second imaging mode is equal to that of the first imaging mode; furthermore, the number of times the same scanning position is scanned in each frame under the second imaging mode is greater than that under the first imaging mode; further, their scanning densities may be the same or different; additionally, when their scanning densities are the same, the scanning power corresponding to the same scanning position in each scan frame under the second imaging mode is equal to that under the first imaging mode, thus their per-frame scanning power is also the same.

For example, the second scanning parameter and the first scanning parameter are configured such that: the scanning range of the second imaging mode is less than that of the first imaging mode, and the number of times the scanning position is scanned in each frame under the second imaging mode is greater than that under the first imaging mode.

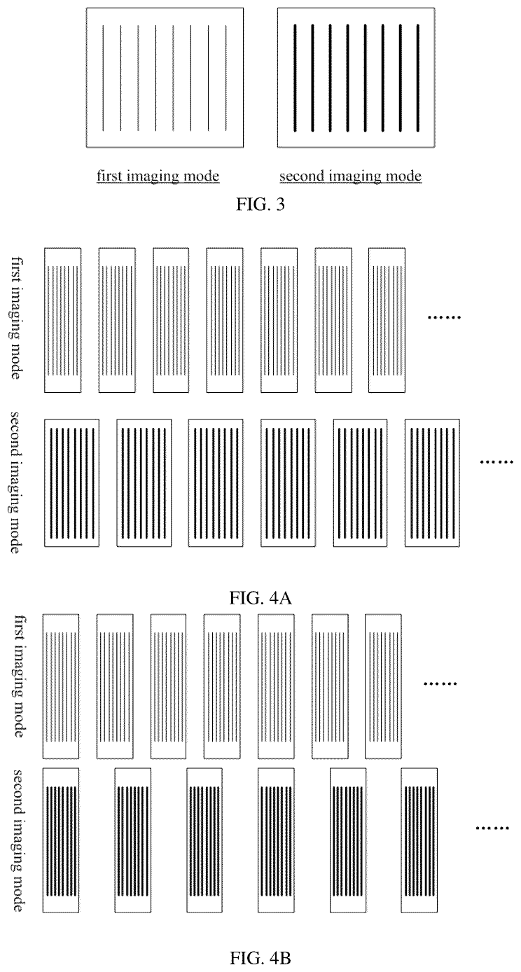

FIG. 4A shows an example of the first imaging mode and the second imaging mode (in full-real-time scanning mode) being scanned. The boxes in the figure represent the scanning corresponding to one frame of ultrasound image, and also represent one scan frame. The time taken for scanning each scan frame under the second imaging mode is greater than that under the first imaging mode, and the time interval between adjacent scan frames under the second imaging mode is equal to that under the first imaging mode. This makes the scanning frame rate of the second imaging mode less than that of the first imaging mode. However, at the same time, the scanning power of each scan frame under the second imaging mode is greater than that under the first imaging mode. For example, their scan line densities are the same, but the scanning power of each scan line in the former is greater than that of the corresponding scan line in the latter.

FIG. 4B shows an example of the first imaging mode and the second imaging mode (in full-real-time scanning mode) being scanned. The boxes in the figure represent the scanning corresponding to one frame of ultrasound image, and also represent one scan frame. The time taken for scanning each scan frame under the second imaging mode is equal to that under the first imaging mode, and the time interval between adjacent scan frames under the second imaging mode is greater than that under the first imaging mode. This makes the scanning frame rate of the second imaging mode less than that of the first imaging mode. However, at the same time, the scanning power of each scan frame under the second imaging mode is greater than that under the first imaging mode. For example, their scan line densities are the same, but the scanning power of each scan line in the former is greater than that of the corresponding scan line in the latter.

FIG. 5 shows an example of the first imaging mode and the second imaging mode (in full-real-time scanning mode) being scanned. The solid boxes in the figure represent the scanning corresponding to one frame of ultrasound image, and the dashed boxes represent the scanning of the imaging region corresponding to one frame of ultrasound image, i.e., the dashed boxes represent one scan frame. Under the second imaging mode, a frame of ultrasound image is synthesized by scanning multiple frames (two frames in the figure) of the imaging region. Taking as an example that scanning a complete imaging region requires 8 scan lines scanned from left to right, synthesizing one frame of ultrasound image by scanning two frames of the imaging region under the second imaging mode can be done as follows: first, scanning these 8 scan lines from left to right, and then scanning the same 8 scan lines from left to right again, such that the scanning position corresponding to each scan line is scanned twice; or scanning each scan line twice, and then proceeding to the next scan line until the last scan line is scanned.

In some embodiments, the second scanning parameter is configured such that the ultrasound imaging system 100 meets a safety index when in operation. The safety index includes at least a mechanical index MI, a temperature, or a thermal index TI.

The above are some explanations of the second imaging mode in comparison with the first imaging mode.

When the scanning mode for the second imaging mode is the semi-real-time scanning mode, step 134 is executed: performing scanning and imaging in this mode.

In some embodiments, step 134 involves alternating between performing a scanning action during a scanning period and performing a first-type action during a pause period. The scanning action includes controlling the ultrasound probe 10 to sequentially scan multiple frames of ultrasound images within the scanning period with a third scanning parameter, wherein upon completion of scanning one frame of ultrasound image, said frame is displayed. The first-type action includes pausing the scanning of ultrasound images during the pause period, and automatically switching from the pause period to the scanning period when the duration of the pause period is reached. In some embodiments, the first-type action also includes freezing and/or replaying the ultrasound image during the pause period.

In some embodiments, selecting the current imaging mode from the first imaging mode and the second imaging mode based on a tissue region, and determining the second imaging mode as the current imaging mode when the tissue region comprises blood vessel, abdomen or craniocerebral.

In some embodiments, the third scanning parameter and the first scanning parameter are configured such that an average scanning energy required to form one frame of ultrasound image under the second imaging mode is greater than that required under the first imaging mode for at least one frame.

In some embodiments, the third scanning parameter and the first scanning parameter are configured such that the average scanning energy required to form one frame of ultrasound image under the second imaging mode is greater than that under the first imaging mode. The average scanning energy of the imaging region has been described above and will not be repeated here.

In some embodiments, the third scanning parameter and the first scanning parameter are further configured such that one or more of the following is satisfied:
(1) The scanning frame rate during the scanning period in the semi-real-time scanning mode under the second imaging mode is less than or equal to the scanning frame rate under the first imaging mode;
(2) The scanning range of the second imaging mode is less than or equal to the scanning range of the first imaging mode;
(3) The scanning density of the second imaging mode is greater than or equal to the scanning density of the first imaging mode;
(4) The number of times the same scanning position is scanned in each frame under the second imaging mode is greater than the number of times the same scanning position is scanned in each frame under the first imaging mode;
(5) The scanning power of each scan frame under the second imaging mode is greater than or equal to the scanning power of each scan frame under the first imaging mode;
(6) The scanning power corresponding to the same scanning position in each scan frame under the second imaging mode is greater than the scanning power corresponding to the same scanning position in each scan frame under the first imaging mode;
(7) The average scanning power per unit time under the second imaging mode is greater than or equal to the average scanning power per unit time under the first imaging mode.

For item (4) above, the number of times the same scanning position is scanned in each ultrasound image of the second imaging mode is greater than that in each ultrasound image of the first imaging mode. Further, their scanning range may also be the same, and the scanning density may also be the same. The scanning position can be a scan point or a scan line. Taking the scan line as an example, if scanning one ultrasound image requires 8 scan lines to be scanned from left to right: under the first imaging mode, a single left-to-right scan is performed, with each scan line scanned once to generate one ultrasound image; under the second imaging mode, multiple left-to-right scans can be performed (so each scan line is scanned multiple times), or scanning can start with the first scan line, each line is scanned multiple times before moving to the next (which is also scanned multiple times), until the last scan line is completed, and then the data is synthesized into one ultrasound image.

For item (6) above, the scanning power corresponding to the same scanning position in each scan frame under the second imaging mode is greater than that in each scan frame under the first imaging mode. Further, their scanning range can be the same, and their scanning density may also be the same. The scanning position can be a scan point or a scan line. The example in FIG. 3 above serves as an illustration and will not be repeated here.

The above situations can be adaptively combined based on their mutual relationships.

For example, the third scanning parameter and the first scanning parameter are configured such that: the scanning frame rate during the scanning period in the semi-real-time scanning mode under the second imaging mode is less than the scanning frame rate under the first imaging mode, and the scanning range of the second imaging mode is less than or equal to that of the first imaging mode; furthermore, the scanning power of each scan frame under the second imaging mode is greater than that under the first imaging mode, for example, their scanning ranges, as well as scanning densities, may be the same, but the scanning power corresponding to the same scanning position in each scan frame under the second imaging mode is greater than that under the first imaging mode; additionally, the time taken for each scan frame under the second imaging mode is greater than that under the first imaging mode, and the time interval between adjacent scan frames under the second imaging mode is greater than that under the first imaging mode; or, the time taken for each scan frame under the second imaging mode is greater than that under the first imaging mode, and the time interval between adjacent scan frames under the second imaging mode is equal to that under the first imaging mode; or, the time taken for each scan frame under the second imaging mode is equal to that under the first imaging mode, and the time interval between adjacent scan frames under the second imaging mode is greater than that under the first imaging mode.

For example, the second scanning parameter and the first scanning parameter are configured such that: the scanning frame rate during the scanning period in the semi-real-time scanning mode under the second imaging mode is less than the scanning frame rate under the first imaging mode, and the scanning range of the second imaging mode is equal to that of the first imaging mode; furthermore, the number of times the same scanning position is scanned in each frame under the second imaging mode is greater than that under the first imaging mode; additionally, their scanning densities may be the same or different; additionally, when their scanning densities are the same, the scanning power corresponding to the same scanning position in each scan frame under the second imaging mode is equal to that under the first imaging mode, thus their per-frame scanning power is also the same.

For example, the third scanning parameter and the first scanning parameter are configured such that: the scanning frame rate during the scanning period in the semi-real-time scanning mode under the second imaging mode is less than the scanning frame rate under the first imaging mode, and the number of times the same scanning position is scanned in each frame under the second imaging mode is greater than the number of times the same scanning position is scanned in each frame under the first imaging mode, wherein the scanning position includes a scan point or a scan line.

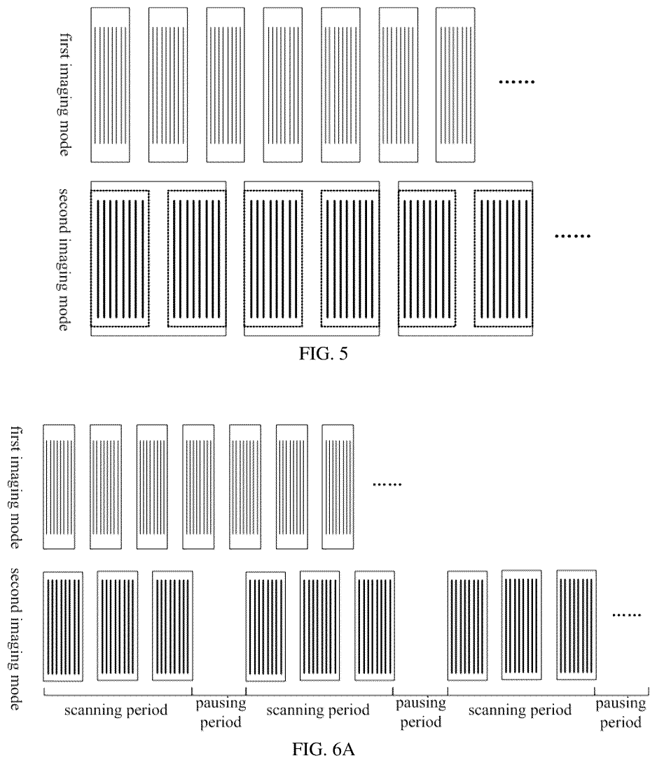

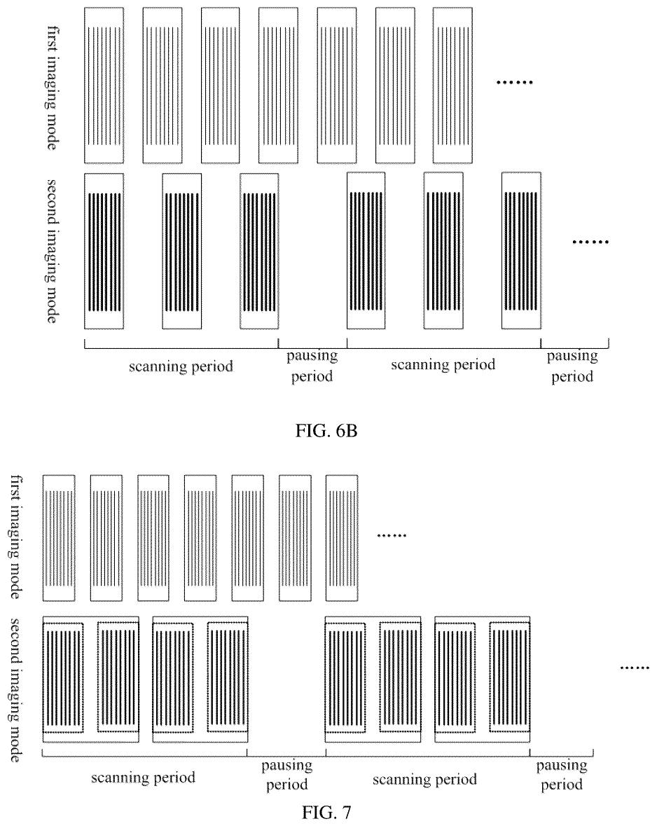

FIG. 6A shows an example of the first imaging mode and the second imaging mode (in semi-real-time scanning mode) being scanned. The boxes in the figure represent the scanning corresponding to one frame of ultrasound image, and also represent one scan frame. In the semi-real-time scanning of the second imaging mode, the scanning period in which a scanning action is performed and the pausing period in which scanning is paused are alternated, the time taken for scanning each scan frame under the second imaging mode is greater than the time taken for scanning each scan frame under the first imaging mode, and the time interval between adjacent scan frames within the scanning period under the second imaging mode is equal to that under the first imaging mode. This makes the scanning frame rate of the second imaging mode in the semi-real-time scanning mode less than the scanning frame rate of the first imaging mode. However, at the same time, the scanning power of each scan frame under the second imaging mode is greater than that under the first imaging mode. For example, their scan line densities are the same, but the scanning power of each scan line in the former is greater than that of the corresponding scan line in the latter.

FIG. 6B shows an example of the first imaging mode and the second imaging mode (in semi-real-time scanning mode) being scanned. The boxes in the figure represent the scanning corresponding to one frame of ultrasound image, and also represent one scan frame. In the semi-real-time scanning of the second imaging mode, the scanning period in which a scanning action is performed and the pausing period in which scanning is paused are alternated, the time taken for scanning each scan frame under the second imaging mode is equal to the time taken for scanning each scan frame under the first imaging mode, and the time interval between adjacent scan frames within the scanning period under the second imaging mode is greater than that under the first imaging mode. This makes the scanning frame rate of the second imaging mode in the semi-real-time scanning mode less than the scanning frame rate of the first imaging mode. However, at the same time, the scanning power of each scan frame under the second imaging mode is greater than that under the first imaging mode. For example, their scan line densities are the same, but the scanning power of each scan line in the former is greater than that of the corresponding scan line in the latter.

FIG. 7 shows an example of the first imaging mode and the second imaging mode (in semi-real-time scanning mode) being scanned. The solid boxes in the figure represent the scanning corresponding to one frame of ultrasound image, and the dashed boxes represent the scanning of the imaging region corresponding to one frame of ultrasound image, i.e., the dashed boxes represent one scan frame. In the semi-real-time scanning of the second imaging mode, the scanning period in which a scanning action is performed and the pausing period in which scanning is paused are alternated. Under the second imaging mode, a frame of ultrasound image is synthesized by scanning multiple frames (two frames in the figure) of the imaging region. Taking as an example that scanning a complete imaging region requires 8 scan lines scanned from left to right, synthesizing one frame of ultrasound image by scanning two frames of the imaging region under the second imaging mode can be done as follows: first, scanning these 8 scan lines from left to right, and then scanning the same 8 scan lines from left to right again, such that the scanning position corresponding to each scan line is scanned twice; or scanning each scan line twice, and then proceeding to the next scan line until the last scan line is scanned.

In some embodiments, the third scanning parameter is configured such that the ultrasound imaging system 100 meets a safety index when in operation. The safety index includes at least a mechanical index MI, a temperature, or a thermal index TI.

The above are some explanations of the second imaging mode in comparison with the first imaging mode.

The following presents a comparative description of the full-real-time scanning mode and the semi-real-time scanning mode under the second imaging mode.

In some embodiments, the time interval between any two frames of ultrasound images scanned in the full-real-time scanning mode is shorter than the duration of the pause period in the semi-real-time scanning mode.

In some embodiments, the second scanning parameter and the third scanning parameter are configured such that one or more of the following are satisfied:

Within the second imaging mode, the scanning frame rate under the full-real-time scanning mode is less than or equal to the frame rate of the scanning period under the semi-real-time scanning mode;

Within the second imaging mode, the scanning range under the full-real-time scanning mode is less than or equal to the scanning range of the scanning period under the semi-real-time scanning mode;

Within the second imaging mode, the scanning power of each scan frame under the full-real-time scanning mode is less than or equal to the scanning power of each scan frame in the scanning period under the semi-real-time scanning mode;

Within the second imaging mode, the average scanning energy required to form one frame of ultrasound image under the full-real-time scanning mode is less than or equal to the average scanning energy required to form one frame of ultrasound image under the semi-real-time scanning mode; for example, when the imaging region or imaging range and the scan line density are the same for both, the scanning power of each scan line in the full-real-time scanning mode is less than or equal to the scanning power of the corresponding scan line in the semi-real-time scanning mode; as another example, when the imaging region or imaging range is the same but the scan line density is different for both, the total power of each frame scan in the full-real-time scanning mode is less than or equal to the total power of each frame scan in the semi-real-time scanning mode;

Within the second imaging mode, the average scanning power per unit time in the full-real-time scanning mode is less than or equal to the average scanning power per unit time in the semi-real-time scanning mode.

Generally, in terms of the imaging quality or signal-to-noise ratio of the ultrasound images: the second imaging mode is superior to the first imaging mode, and the semi-real-time scanning mode under the second imaging mode is superior to the full-real-time scanning mode under the second imaging mode.

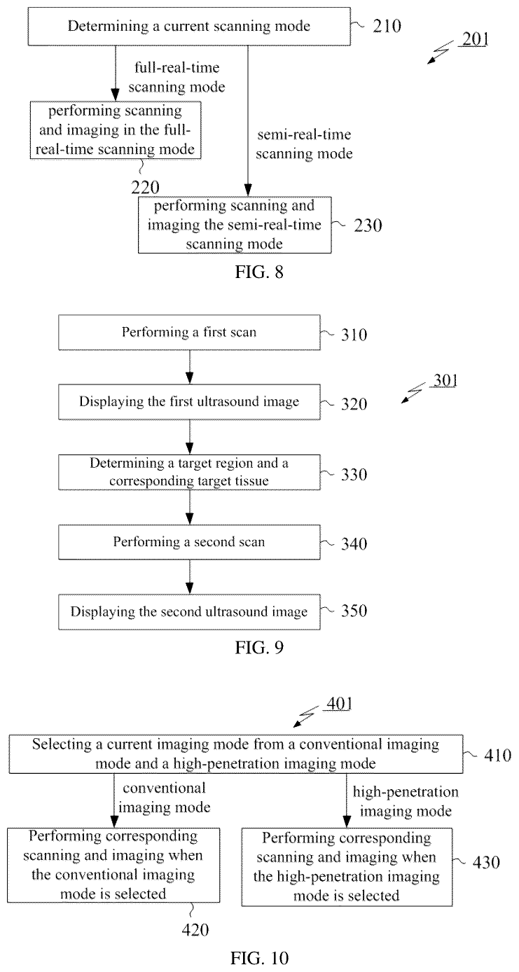

Referring to FIG. 8, an ultrasound imaging method 201 is disclosed in some embodiments, which can be applied to the ultrasound imaging system 100 of any embodiment of the present application. In some embodiments, the ultrasound imaging method 201 includes the following steps:

Step 210: determining a current scanning mode.

In some embodiments, the scanning mode includes a full-real-time scanning mode and a semi-real-time scanning mode.

The ultrasound imaging system 100 can automatically determine the scanning mode, or can manually select the scanning mode based on user operation.

In some embodiments, step 210 involves acquiring a current scanning item, determining the full-real-time scanning mode as the current scanning mode when the current scanning item is a first-type scanning item, and determining the semi-real-time scanning mode as the current scanning mode when the current scanning item is a second-type scanning item.

Scanning items are generally defined for a scanned region, such as heart scan, liver scan, abdominal scan, kidney scan, thyroid scan, etc. Scanning items can be pre-classified into the first-type scanning items and the second-type scanning items. The classification may be based on the number of slice types to be scanned in the scanning protocol corresponding to a scanning item: if the number is large (e.g., greater than a first number), it is categorized as the first-type scanning item; and if the number is small (e.g., less than a second number), it is categorized as the second-type scanning item, where the first number is greater than or equal to the second number. Another basis for classification may be the depth of the scanned region from the scanning body surface: if the depth is less than a first depth, it is categorized as the first-type scanning item, and if the depth is greater than the second depth, it is categorized as the second-type scanning item, where the second depth is greater than or equal to the first depth.

In some embodiments, step 210 involves recognizing the scanned region based on the current ultrasound image, determining the full-real-time scanning mode as the current scanning mode when the current scanned region is a first-type tissue region, and determining the semi-real-time scanning mode as the current scanning mode when the current scanned region is a second-type tissue region. Step 210 may involve recognizing the scanned region based on the ultrasound image using traditional image recognition or machine learning.

In some embodiments, step 210 involves acquiring the number of currently pending slice types to be scanned, determining the full-real-time scanning mode as the current scanning mode when the number is greater than the first number, and determining the semi-real-time scanning mode as the current scanning mode when the number is less than the second number; wherein the first number is greater than or equal to the second number.

In some embodiments, step 210 involves acquiring a current imaging depth, determining the full-real-time scanning mode as the current scanning mode when the current imaging depth is less than a first depth threshold, and determining the semi-real-time scanning mode as the current scanning mode when the current imaging depth is greater than a second depth threshold. In some embodiments, the second depth threshold is greater than or equal to the first depth threshold.

In some embodiments, step 210 involves acquiring the depth of a target region from the ultrasound image, determining the full-real-time scanning mode as the current scanning mode when the depth of the target region is less than a third depth threshold, and determining the semi-real-time scanning mode as the current scanning mode when the depth of the target region is greater than a fourth depth threshold; wherein the fourth depth threshold is greater than or equal to the third depth threshold. In some examples, the target region includes one of the following: a region selected by a user in the ultrasound image, and a lesion region recognized from the ultrasound image.

When the current scanning mode is the full-real-time scanning mode, step 220 is executed: performing scanning and imaging in this mode.

In some embodiments, step 220 involves controlling the ultrasound probe 10 to sequentially scan multiple frames of ultrasound images with the second scanning parameter; wherein, upon completion of scanning one frame of ultrasound image, said frame is displayed.

Further explanations for step 220 can be found in the foregoing descriptions of step 132, and will not be repeated here.

When the scanning mode for the second imaging mode is the semi-real-time scanning mode, step 230 is executed: performing scanning and imaging in this mode.

In some embodiments, step 230 involves alternating between performing a scanning action during a scanning period and performing a first-type action during a pause period. The scanning action includes controlling the ultrasound probe 10 to sequentially scan multiple frames of ultrasound images within the scanning period with a third scanning parameter, wherein upon completion of scanning one frame of ultrasound image, said frame is displayed. The first-type action includes pausing the scanning of ultrasound images during the pause period, and automatically switching from the pause period to the scanning period when the duration of the pause period is reached. In some embodiments, the first-type action also includes freezing and/or replaying the ultrasound image during the pause period.

Further explanations for step 230 can be found in the foregoing descriptions of step 134, and will not be repeated here.

In some embodiments, the time interval between any two frames of ultrasound images scanned under the full-real-time scanning mode is shorter than the duration of the pause period under the semi-real-time scanning mode.

In some embodiments, the scanning frame rate under the full-real-time scanning mode is less than or equal to the scanning frame rate of the scanning period under the semi-real-time scanning mode.

In some embodiments, the scanning range under the full-real-time scanning mode is less than or equal to the scanning range in the scanning period under the semi-real-time scanning mode.

In some embodiments, the average scanning power per unit time under the full-real-time scanning mode for the second imaging mode is less than or equal to the average scanning power per unit time under the semi-real-time scanning mode for the second imaging mode.

In some embodiments, the scanning power of each scan frame under the full-real-time scanning mode is less than or equal to the scanning power of each scan frame in the scanning period under the semi-real-time scanning mode; for example, when the imaging region or imaging range and the scan line density are the same for both, the scanning power of each scan line in the full-real-time scanning mode is less than or equal to the scanning power of the corresponding scan line in the semi-real-time scanning mode; as another example, when the imaging region or imaging range is the same but the scan line density is different for both, the total power of each scan frame in the full-real-time scanning mode is less than or equal to the total power of each scan frame in the semi-real-time scanning mode.

In some embodiments, an average scanning energy required to form one frame of ultrasound image under the semi-real-time scanning mode is greater than or equal to that required under the full-real-time scanning mode for at least one frame.

In some embodiments, the average scanning energy required to form one frame of ultrasound image under the full-real-time scanning mode is less than or equal to the average scanning energy required to form one frame of ultrasound image under the semi-real-time scanning mode.

Generally, in terms of the imaging quality or signal-to-noise ratio of the ultrasound images: the semi-real-time scanning mode under the second imaging mode is superior to the full-real-time scanning mode under the second imaging mode.

Some embodiments propose two scanning modes, namely the full-real-time scanning mode and the semi-real-time scanning mode. Different scanning modes may be selected according to specific scenarios to meet the imaging quality requirements.

Referring to FIG. 9, some embodiments disclose an ultrasound imaging method 301, which can be applied to the ultrasound imaging system 100 of any embodiment of the present disclosure. In some embodiments, the ultrasound imaging method 301 includes the following steps:

Step 310: Performing a first scan.

In some embodiments, step 310 involves controlling the ultrasound probe 10 to scan a tissue to be imaged with a first scanning parameter, thereby obtaining a first ultrasound image of the tissue to be imaged.

Step 320: Displaying the first ultrasound image.

In some embodiments, step 320 involves displaying the first ultrasound image in a first region of the display interface.

Step 330: Determining a target region and a corresponding target tissue.

In some embodiments, step 330 involves determining the target region based on the first ultrasound image and determines the target tissue corresponding to the target region in the tissue to be imaged.

In some examples, step 330 involves receiving a region selection operation by a user on the first ultrasound image, determining the region corresponding to the region selection operation on the first ultrasound image, and taking it as the target region.

In some examples, step 330 involves recognizing the first ultrasound image to obtain a recognition result, and determining the target region on the first ultrasound image based on the recognition result. In some examples, the recognition result includes a target anatomical structure or a lesion structure, so that the target region is a region containing the target anatomical structure or the lesion structure.

Step 340: Performing a second scan.

In some embodiments, step 340 involves controlling the ultrasound probe 10 to scan the target tissue with a second scanning parameter, thereby obtaining a second ultrasound image containing the target tissue.
Step 350: Displaying the second ultrasound image.

In some embodiments, step 350 involves displaying the second ultrasound image in the first region of the display interface to replace the first ultrasound image.

In some embodiments, the scanning density of the target tissue in the second scan is greater than that in the first scan; wherein the scanning density includes a point density or a line density.

In some embodiments, the number of times each scanning position in each frame of the target tissue is scanned in the second scan is greater than that in the first scan; wherein the scanning position includes a scan point or a scan line.

In some embodiments, the scanning power of each scan frame of the target tissue in the second scan is greater than the scanning power of each scan frame of the target tissue in the first scan.

In some embodiments, the scanning power corresponding to the same scanning position of the target tissue in each scan frame in the second scan is greater than that in the first scan; further, the scanning densities of the target tissue in both scans may be the same.

In some embodiments, an average scanning energy required to form one frame of ultrasound image in the second scan is greater than or equal to that required in the first scan for at least one frame.

In some embodiments, the average scanning energy required to form one frame of ultrasound image in the second scan is greater than or equal to that in the first scan. The description of the average scanning energy required to form one frame of ultrasound image can be found in the foregoing, and will not be repeated here.

In some embodiments, an display characteristic of the first ultrasound image is higher than the display characteristic of the second ultrasound image, and the display characteristic comprises at least one of an image penetration, an image clarity and a richness of image details.

In some examples, the first ultrasound image and the second ultrasound image are of the same type, such as both being B-mode images.

By determining the target region and the target tissue from the first ultrasound image and reducing the scanning region to the target tissue corresponding to the target region, the imaging quality of the target region can be improved while the ultrasound imaging system 100 also meets safety standards during operation.

Referring to FIG. 10, some embodiments disclose an ultrasound imaging method 401, which can be applied to the ultrasound imaging system 100 of any embodiment of the present disclosure. In some embodiments, the ultrasound imaging method 401 includes the following steps:

Step 410: Selecting a current imaging mode from a conventional imaging mode and a high-penetration imaging mode.

The ultrasound imaging system 100 can automatically select the current imaging mode from the conventional imaging mode and the high-penetration imaging mode, or can manually select the current imaging mode from the conventional imaging mode and the high-penetration imaging mode based on user operation.

In some embodiments, step 410 involves acquiring the depth of the target region from the ultrasound image, selecting the high-penetration imaging mode as the current imaging mode when the depth of the target region is greater than or equal to a target depth threshold. In some examples, the target region includes any one of: a region selected by a user in the ultrasound image, or a lesion region recognized from the ultrasound image.

In some embodiments, step 410 involves calculating a signal intensity obtained by scanning in the conventional imaging mode, and switching the imaging mode from the conventional imaging mode to the high-penetration imaging mode when the signal intensity is less than a signal intensity threshold.

In some embodiments, step 410 involves calculating the signal-to-noise ratio of a signal obtained by scanning under the conventional imaging mode, and switching the imaging mode is switched from the conventional imaging mode to the high-penetration imaging mode when the signal-to-noise ratio is less than a ratio threshold.

Step 420: Performing corresponding scanning and imaging when the conventional imaging mode is selected.

In some embodiments, when the conventional imaging mode is selected as the current imaging mode, step 420 involves controlling the ultrasound probe 10 to sequentially scan multiple frames of ultrasound images with a first scanning parameter; wherein upon completion of scanning one frame of ultrasound image, said frame is displayed.

In some examples, the first scanning parameter is configured such that the ultrasound imaging system 100 meets a safety index when in operation. The safety index includes at least a mechanical index MI, a temperature, or a thermal index TI.

Step 430: Performing corresponding scanning and imaging when the high-penetration imaging mode is selected.

In some embodiments, when the high-penetration imaging mode is selected as the current imaging mode, step 430 involves controlling the ultrasound probe 10 to sequentially scan multiple frames of ultrasound images with a second scanning parameter; where upon completion of scanning one frame of ultrasound image, said frame is displayed.

In some examples, the second scanning parameter is configured such that the ultrasound imaging system 100 meets a safety index when in operation. The safety index includes at least a mechanical index MI, a temperature, or a thermal index TI.

In some embodiments, the second scanning parameter and the first scanning parameter are configured such that any combination of the following is satisfied the following:
(1) The scanning density within a target scanning range under the high-penetration imaging mode is higher than that under the conventional imaging mode;
(2) The scanning density within a non-target scanning range under the high-penetration imaging mode is lower than that under the conventional imaging mode;
(3) The number of times the same scanning position within the target scanning range in each frame is scanned under the high-penetration imaging mode is greater than that under the conventional imaging mode;
(4) The number of times the same scanning position within the non-target scanning range in each frame is scanned under the high-penetration imaging mode is lower than that under the conventional imaging mode;
(5) The scanning range under the high-penetration imaging mode is smaller than that under the conventional imaging mode;
(6) The scanning frame rate under the high-penetration imaging mode is lower than that under the conventional imaging mode;
(7) The transmission frequency under the high-penetration imaging mode is lower than that under the conventional imaging mode;
(8) The scanning power corresponding to the same scanning position in each scan frame under the high-penetration imaging mode is higher than that under the conventional imaging mode, wherein the scanning power includes an emission power corresponding to a scan point or an emission power corresponding to a scan line.

The above involves the target scanning range and the non-target scanning range. The target scanning range may be determined in the following way: it may be determined based on a region selected by a user in the ultrasound image, and accordingly, the other regions in the ultrasound image or the unselected regions are the non-target scanning range; it may also be determined based on the lesion region recognized in the ultrasound image as the target scanning range, and the other regions in the ultrasound image are the non-target scanning range.

In some embodiments, an display characteristic of a ultrasound image under the high-penetration imaging mode is higher than a ultrasound image under the conventional imaging mode, and the display characteristic comprises at least one of an image penetration, an image clarity and a richness of image details, such as the ultrasound image is about the same tissue region under the high-penetration imaging mode and the conventional imaging mode.

In some embodiments, the second scanning parameter and the first scanning parameter are further configured such that one or more of the following is satisfied:

The scanning frame rate under the high-penetration imaging mode is less than or equal to the scanning frame rate under the conventional imaging mode;

The scanning range under the high-penetration imaging mode is less than or equal to the scanning range under the conventional imaging mode;

The scanning density under the high-penetration imaging mode is greater than or equal to the scanning density under the conventional imaging mode;

The number of times the same scanning position is scanned in each frame under the high-penetration imaging mode is greater than the number of times the same scanning position is scanned in each frame under the conventional imaging mode;

The scanning power of each scan frame under the high-penetration imaging mode is greater than or equal to the scanning power of each scan frame under the conventional imaging mode;

The scanning power corresponding to the same scanning position in each scan frame under the high-penetration imaging mode is greater than the scanning power corresponding to the same scanning position in each scan frame under the conventional imaging mode;

The average scanning power per unit time under the high-penetration imaging mode is greater than or equal to the average scanning power per unit time under the conventional imaging mode.

For example, the second scanning parameter and the first scanning parameter are configured such that: the scanning frame rate under the high-penetration imaging mode is less than that under the conventional imaging mode, and the scanning range under the high-penetration imaging mode is less than or equal to that under the conventional imaging mode; furthermore, the scanning power of each scan frame under the high-penetration imaging mode is greater than that under the conventional imaging mode for example, their scanning densities may be the same, but the scanning power corresponding to the same scanning position in each scan frame under the high-penetration imaging mode is greater than that under the conventional imaging mode; additionally, the time taken for each scan frame under the high-penetration imaging mode is greater than that under the conventional imaging mode, and the time interval between adjacent scan frames under the high-penetration imaging mode is greater than that under the conventional imaging mode; or, the time taken for each scan frame under the high-penetration imaging mode is greater than that under the conventional imaging mode, and the time interval between adjacent scan frames under the high-penetration imaging mode is equal to that under the conventional imaging mode; or, the time taken for each scan frame under the high-penetration imaging mode is equal to that under the conventional imaging mode, and the time interval between adjacent scan frames under the high-penetration imaging mode is greater than that under the conventional imaging mode.

For example, the second scanning parameter and the first scanning parameter are configured such that: the scanning frame rate under the high-penetration imaging mode is less than that under the conventional imaging mode, and the scanning range under the high-penetration imaging mode is equal to that under the conventional imaging mode; furthermore, the number of times the same scanning position is scanned in each frame under the high-penetration imaging mode is greater than that under the conventional imaging mode; further, their scanning densities may be the same or different; additionally, their scanning densities are the same, the scanning power corresponding to the same scanning position in each scan frame under the high-penetration imaging mode is equal to that under the conventional imaging mode, thus their per-frame scanning power is also the same.

For example, the second scanning parameter and the first scanning parameter are configured such that: the scanning range under the high-penetration imaging mode is less than that under the conventional imaging mode, and the number of times the scanning position is scanned in each frame under the high-penetration imaging mode is greater than that under the conventional imaging mode.

When the scanning frame rate under the high-penetration imaging mode is lower than that under the conventional imaging mode, it can be achieved through one or more scanning modes.

In some embodiments, under the high-penetration imaging mode, the ultrasound probe 10 is controlled with the second scanning parameter to scan multiple frames of ultrasound images in sequence, this scanning method may be referred to as a first scanning method; wherein, upon completion of scanning one frame of ultrasound image, said frame is displayed.

In some examples, the second scanning parameter and the first scanning parameter are configured such that an average scanning energy required to form one frame of ultrasound image under the high-penetration imaging mode is greater than that required under the conventional imaging mode for at least one frame.

In some embodiments, the second scanning parameter and the first scanning parameter are configured such that an average scanning energy required to form one frame of ultrasound image under the high-penetration imaging mode is greater than that required under the conventional imaging mode for at least one frame.

In some embodiments, under the high-penetration imaging mode, performing a scanning action during a scanning period and performing a first-type action during a pause period are alternated. The scanning action includes controlling the ultrasound probe 10 to sequentially scan multiple frames of ultrasound images within the scanning period with a second scanning parameter, this scanning method may be referred to as a second scanning method; wherein upon completion of scanning one frame of ultrasound image, said frame is displayed. The first-type action includes pausing the scanning of ultrasound images during the pause period, and automatically switching from the pause period to the scanning period when the duration of the pause period is reached. In some embodiments, the first-type action also includes freezing and/or replaying the ultrasound image during the pause period. In some embodiments, the second scanning parameter and the first scanning parameter are configured such that an average scanning energy required to form one frame of ultrasound image under the high-penetration imaging mode is greater than the average scanning energy required to form one frame of ultrasound image under the conventional imaging mode.

In some embodiments, the ultrasound imaging system 100 can automatically determine the scanning mode, or can manually select the scanning mode based on user operation.

In some embodiments, under the high-penetration imaging mode, a current scanning item is acquired, the full-real-time scanning mode is determined as the current scanning mode when the current scanning item is a first-type scanning item, and the semi-real-time scanning mode is determined as the current scanning mode when the current scanning item is a second-type scanning item.

Scanning items are generally defined for a scanned region, such as heart scan, liver scan, abdominal scan, kidney scan, thyroid scan, etc. Scanning items can be pre-classified into the first-type scanning items and the second-type scanning items. The classification may be based on the number of slice types to be scanned in the scanning protocol corresponding to a scanning item: if the number is large (e.g., greater than a first number), it is categorized as the first-type scanning item; and if the number is small (e.g., less than a second number), it is categorized as the second-type scanning item, where the first number is greater than or equal to the second number. Another basis for classification may be the depth of the scanned region from the scanning body surface: if the depth is less than a first depth, it is categorized as the first-type scanning item, and if the depth is greater than the second depth, it is categorized as the second-type scanning item, where the second depth is greater than or equal to the first depth.

In some embodiments, under the high-penetration imaging mode, the scanned region is recognized based on the current ultrasound image, the full-real-time scanning mode is determined as the current scanning mode when the current scanned region is a first-type tissue region, and the semi-real-time scanning mode is determined as the current scanning mode when the current scanned region is a second-type tissue region. Step 410 may involve recognizing the scanned region based on the ultrasound image using traditional image recognition or machine learning.

In some embodiments, under the high-penetration imaging mode, the number of currently pending slice types to be scanned is acquired, the full-real-time scanning mode is determined as the current scanning mode when the number is greater than the first number, and the semi-real-time scanning mode is determined as the current scanning mode when the number is less than the second number; wherein the first number is greater than or equal to the second number.

In some embodiments, under the high-penetration imaging mode, a current imaging depth is acquired, the full-real-time scanning mode is determined as the current scanning mode when the current imaging depth is less than a first depth threshold, and the semi-real-time scanning mode is determined as the current scanning mode when the current imaging depth is greater than a second depth threshold. In some embodiments, the second depth threshold is greater than or equal to the first depth threshold.

In some embodiments, under the high-penetration imaging mode, the depth of a target region from the ultrasound image is acquired, the full-real-time scanning mode is determined as the current scanning mode when the depth of the target region is less than a third depth threshold, and the semi-real-time scanning mode is determined as the current scanning mode when the depth of the target region is greater than a fourth depth threshold; wherein the fourth depth threshold is greater than or equal to the third depth threshold. In some examples, the target region includes one of the following: a region selected by a user in the ultrasound image, and a lesion region recognized from the ultrasound image.

The above methods can make the penetration ability under the high-penetration imaging mode greater than that under the conventional imaging mode, thereby enhancing the intensity of the ultrasound echo signals.

Some solutions, for example, propose a conventional imaging mode and a high-penetration imaging mode. Different imaging modes can be selected according to different situations. While meeting the imaging quality requirements, the ultrasound imaging system 100 also complies with safety standards when in operation.

The present disclosure is described with reference to various exemplary embodiments. However, those skilled in the art will recognize that changes and modifications can be made to the exemplary embodiments without departing from the scope of this document. For example, various operation steps and components used to perform the operation steps can be implemented in different ways depending on specific applications or any cost functions associated with the operation of the system (for example, one or more steps can be deleted, modified, or combined with other steps).

In the above embodiments, all or part can be implemented through software, hardware, firmware, or any combination thereof. Additionally, as understood by those skilled in the art, the principles of the present disclosure can be reflected in a computer program product on a computer-readable storage medium, which is pre-installed with computer-readable program code. Any tangible, non-transitory computer-readable storage medium can be used, including magnetic storage devices (hard disks, floppy disks, etc.), optical storage devices (CD-ROMs, DVDs, Blu-ray discs, etc.), flash memory, and the like. These computer program instructions can be loaded onto a general-purpose computer, a special-purpose computer, or other programmable data processing device to form a machine, such that the instructions executed on the computer or other programmable data processing device can generate a device that performs the specified functions. These computer program instructions can also be stored in a computer-readable memory, which can direct a computer or other programmable data processing device to operate in a specific manner, so that the instructions stored in the computer-readable memory can form an article of manufacture, including an implementation device that performs the specified functions. Computer program instructions can also be loaded onto a computer or other programmable data processing device to execute a series of operation steps to produce a computer-implemented process, such that the instructions executed on the computer or other programmable device can provide steps for performing the specified functions.

Although the principles of the present disclosure have been shown in various embodiments, many modifications to the structure, arrangement, proportion, components, materials, and parts that are particularly suitable for specific environments and operational requirements can be made without departing from the principles and scope of the present disclosure. Such modifications and other changes or corrections will be included within the scope of the present disclosure.

The foregoing detailed description has been made with reference to various embodiments. However, those skilled in the art will recognize that various modifications and changes can be made without departing from the scope of the present disclosure. Therefore, the consideration of this disclosure will be in an illustrative rather than a restrictive sense, and all such modifications will be included within its scope. Similarly, the advantages, other advantages, and solutions to problems described with respect to various embodiments as described above should not be construed as critical, essential, or necessary. The term “including” and its variants used herein are non-exclusive inclusions, such that a process, method, article, or device including a list of elements not only includes those elements but also includes other elements not specifically listed or not included within the process, method, system, article, or device. Additionally, the term “coupled” and its variants used herein refer to physical connection, electrical connection, magnetic connection, optical connection, communication connection, functional connection, and/or any other connection.

Those skilled in the art will recognize that many changes can be made to the details of the above embodiments without departing from the basic principles of the present disclosure. Therefore, the scope of the present disclosure should be determined only by the claims.

1. An ultrasound imaging method, applicable to an ultrasound imaging system, comprising:
selecting a current imaging mode from a first imaging mode and a second imaging mode;
when the first imaging mode is selected as the current imaging mode, controlling an ultrasound probe to sequentially scan multiple frames of ultrasound images with a first scanning parameter, wherein upon completion of scanning one frame of ultrasound image, said frame is displayed;
when the second imaging mode is selected as the current imaging mode, determining a scanning mode for the second imaging mode, the scanning mode comprising a full-real-time scanning mode and a semi-real-time scanning mode;
when the scanning mode for the second imaging mode is the full-real-time scanning mode, controlling the ultrasound probe to sequentially scan multiple frames of ultrasound images with a second scanning parameter; wherein: upon completion of scanning one frame of ultrasound image, said frame is displayed; and the second scanning parameter and the first scanning parameter are configured such that an average scanning energy required to form one frame of ultrasound image in an imaging region under the second imaging mode is greater than that required under the first imaging mode; and
when the scanning mode for the second imaging mode is the semi-real-time scanning mode, alternately performing a scanning action during a scanning period and a first-type action during a pause period; wherein: the scanning action comprises controlling the ultrasound probe to sequentially scan multiple frames of ultrasound images with a third scanning parameter during the scanning period; upon completion of scanning one frame of ultrasound image, said frame is displayed; the first-type action comprises pausing scanning ultrasound images during the pause period, and switching automatically from the pause period to the scanning period after a duration of the pause period has elapsed; and the third scanning parameter and the first scanning parameter are configured such that an average scanning energy required to form one frame of ultrasound image in an imaging region under the second imaging mode is greater than that required under the first imaging mode.

2. The ultrasound imaging method according to claim 1, wherein the second scanning parameter and the first scanning parameter are further configured such that one or more of the following is satisfied:
a scanning frame rate under the second imaging mode is less than or equal to that under the first imaging mode;
a scanning range under the second imaging mode is less than or equal to that under the first imaging mode;
a scanning density under the second imaging mode is greater than or equal to that under the first imaging mode;
a number of times a same scanning position is scanned in each frame under the second imaging mode is greater than that under the first imaging mode;
a scanning power for each scan frame under the second imaging mode is greater than or equal to that under the first imaging mode;
a scanning power corresponding to a same scanning position in each scan frame under the second imaging mode is greater than that under the first imaging mode;
an average scanning power per unit time under the second imaging mode is greater than or equal to that under the first imaging mode.

3. The ultrasound imaging method according to claim 1, wherein the third scanning parameter and the first scanning parameter are further configured such that one or more of the following is satisfied:
a scanning frame rate during the scanning period in the semi-real-time scanning mode under the second imaging mode is less than or equal to a scanning frame rate under the first imaging mode;
a scanning range under the second imaging mode is less than or equal to that under the first imaging mode;
a scanning density under the second imaging mode is greater than or equal to that under the first imaging mode;
a number of times a same scanning position is scanned in each frame under the second imaging mode is greater than that under the first imaging mode;
a scanning power of each scan frame under the second imaging mode is greater than or equal to that under the first imaging mode;
a scanning power corresponding to a same scanning position in each scan frame under the second imaging mode is greater than that under the first imaging mode;
an average scanning power per unit time under the second imaging mode is greater than or equal to that under the first imaging mode.

4. The ultrasound imaging method according to claim 1, wherein selecting a current imaging mode from a first imaging mode and a second imaging mode comprises one or more of the following:
obtaining a depth of a target region from an ultrasound image, and selecting the second imaging mode as the current imaging mode when the depth of the target region is greater than or equal to a target depth threshold, wherein the target region comprises any one of: a region selected by a user in the ultrasound image, and a lesion region recognized based on the ultrasound image;
calculating a signal intensity obtained by scanning under the first imaging mode, and switching from the first imaging mode to the second imaging mode when the signal intensity is less than a signal intensity threshold;
calculating a signal-to-noise ratio of a signal obtained by scanning under the first imaging mode, and switching from the first imaging mode to the second imaging mode when the signal-to-noise ratio is less than a ratio threshold;
selecting the first imaging mode as the current imaging mode in response to a first instruction input by a user;
selecting the second imaging mode as the current imaging mode in response to a second instruction input by a user.

5. The ultrasound imaging method according to claim 1, wherein the first-type action further comprises: freezing an ultrasound image and/or replaying an ultrasound image during the pause period.

6. The ultrasound imaging method according to claim 1, wherein a time interval between any two frames of ultrasound images scanned under the full-real-time scanning mode is less than the duration of the pause period under the semi-real-time scanning mode.

7. The ultrasound imaging method according to claim 1, wherein the second scanning parameter and the third scanning parameter are configured such that one or more of the following is satisfied:
within the second imaging mode, a scanning frame rate under the full-real-time scanning mode is less than or equal to a scanning frame rate during the scanning period under the semi-real-time scanning mode;
within the second imaging mode, a scanning range under the full-real-time scanning mode is less than or equal to a scanning range during the scanning period under the semi-real-time scanning mode;
within the second imaging mode, a scanning power of each scan frame under the full-real-time scanning mode is less than or equal to a scanning power of each scan frame during the scanning period under the semi-real-time scanning mode;
within the second imaging mode, an average scanning energy required to form one frame of ultrasound image in an imaging region under the full-real-time scanning mode is less than or equal to that under the semi-real-time scanning mode;
within the second imaging mode, an average scanning power per unit time under the full-real-time scanning mode is less than or equal to that under the semi-real-time scanning mode.

8. The ultrasound imaging method according to claim 1, wherein determining a scanning mode for the second imaging mode comprises any one of the following:
obtaining a current scanning item, determining the full-real-time scanning mode as the scanning mode for the second imaging mode when the current scanning item is a first-type scanning item, and determining the semi-real-time scanning mode as the scanning mode for the second imaging mode when the current scanning item is a second-type scanning item;
recognizing a scanned region based on a current ultrasound image, determining the full-real-time scanning mode as the scanning mode for the second imaging mode when a current scanned region is a first-type tissue region, and determining the semi-real-time scanning mode as the scanning mode for the second imaging mode when the current scanned region is a second-type tissue region;
obtaining a number of cross-sectional types to be scanned currently, determining the full-real-time scanning mode as the scanning mode for the second imaging mode when said number is greater than a first number, and determining the semi-real-time scanning mode as the scanning mode for the second imaging mode when said number is less than a second number, wherein the first number is greater than or equal to the second number;
obtaining a current imaging depth, determining the full-real-time scanning mode as the scanning mode for the second imaging mode when the current imaging depth is less than a first depth threshold, and determining the semi-real-time scanning mode as the scanning mode for the second imaging mode when the current imaging depth is greater than a second depth threshold, wherein the second depth threshold is greater than or equal to the first depth threshold;
obtaining a depth of the target region from the ultrasound image, determining the full-real-time scanning mode as the scanning mode for the second imaging mode when the depth of the target region is less than a third depth threshold, and determining the semi-real-time scanning mode as the scanning mode for the second imaging mode when the depth of the target region is greater than a fourth depth threshold; wherein the fourth depth threshold is greater than or equal to the third depth threshold; and the target region includes any one of: a region selected by a user in the ultrasound image, and a lesion region recognized based on the ultrasound image.

9. The ultrasound imaging method according to claim 1, wherein the average scanning energy required to form one frame of ultrasound image is a ratio of a total scanning power required to form one frame of ultrasound image to an area of the imaging region under a corresponding imaging mode, wherein the scanning power comprises an emission power corresponding to a scan point or an emission power corresponding to a scan line.

10. The ultrasound imaging method according to claim 1, wherein the second scanning parameter is configured such that the ultrasound imaging system meets a safety index when working; and/or, the third scanning parameter is configured such that the ultrasound imaging system meets the safety index when working; wherein the safety index comprises at least one of: a mechanical index MI, a temperature, and a thermal index TI.

11. The ultrasound imaging method according to claim 1, wherein selecting the current imaging mode from the first imaging mode and the second imaging mode based on a tissue region, and determining the second imaging mode as the current imaging mode when the tissue region comprises blood vessel, abdomen or craniocerebral.

12. An ultrasound imaging method, comprising:
performing a first scan: controlling an ultrasound probe to scan a target tissue to be imaged with a first scanning parameter to obtain a first ultrasound image of the target tissue to be imaged;
displaying the first ultrasound image in a first region of a display interface;
determining a target region based on the first ultrasound image, and determining, in the target tissue to be imaged, a target tissue corresponding to the target region;
performing a second scan: controlling the ultrasound probe to scan the target tissue with a second scanning parameter to obtain a second ultrasound image including the target tissue; and
displaying the second ultrasound image in the first region of the display interface so as to replace the first ultrasound image.

13. The ultrasound imaging method according to claim 12, wherein the second scanning parameter and the first scanning parameter are further configured such that one or more of the following is satisfied:
a scanning density of the target tissue in the second scan is greater than that in the first scan; the scanning density comprises a point density or a line density;
a number of times a same scanning position is scanned in each frame of the target tissue in the second scan is greater than that in the first scan; the scanning position comprises a scan point or a scan line;
a scanning power of each scan frame of the target tissue in the second scan is greater than that in the first scan;
a scanning power corresponding to a same scanning position of the target tissue in each scan frame in the second scan is greater than that in the first scan;
an average scanning energy required to form one frame of ultrasound image in the second scan is greater than or equal to that in the first scan.

14. The ultrasound imaging method according to claim 12, wherein determining a target region based on the first ultrasound image comprises any one of the following:
receiving a region selection operation by a user on the first ultrasound image, determining a region corresponding to the region selection operation on the first ultrasound image, and taking it as the target region;
performing recognition on the first ultrasound image to obtain a recognition result, and determining the target region on the first ultrasound image based on the recognition result.

15. The ultrasound imaging method according to claim 14, wherein the recognition result comprises a target anatomical structure or a lesion structure.

16. The ultrasound imaging method according to claim 12, wherein an display characteristic of the first ultrasound image is higher than the display characteristic of the second ultrasound image, and the display characteristic comprises at least one of an image penetration, an image clarity and a richness of image details.

17. An ultrasound imaging method, applied to an ultrasound imaging system, comprising:
selecting a current imaging mode from a conventional imaging mode and a high-penetration imaging mode;
when the conventional imaging mode is selected as the current imaging mode, controlling an ultrasound probe to sequentially scan multiple frames of ultrasound images with a first scanning parameter, wherein upon completion of scanning one frame of ultrasound image, said frame is displayed;
when the high-penetration imaging mode is selecting as the current imaging mode, controlling an ultrasound probe to sequentially scan multiple frames of ultrasound images with a second scanning parameter, wherein upon completion of scanning one frame of ultrasound image, said frame is displayed;
the second scanning parameter and the first scanning parameter are configured such that any combination of the following is satisfied:
a scanning density within a target scanning range under the high-penetration imaging mode is higher than that under the conventional imaging mode;
a scanning density within a non-target scanning range under the high-penetration imaging mode is lower than that under the conventional imaging mode;
a number of times a same scanning position is scanned in each frame within the target scanning range under the high-penetration imaging mode is greater than that under the conventional imaging mode;
a number of times a same scanning position is scanned in each frame within the non-target scanning range under the high-penetration imaging mode is lower than that under the conventional imaging mode;
a scanning range under the high-penetration imaging mode is smaller than that under the conventional imaging mode;
a scanning frame rate under the high-penetration imaging mode is lower than that under the conventional imaging mode;
a transmission frequency under the high-penetration imaging mode is lower than that under the conventional imaging mode;
a scanning power corresponding to a same scanning position in each scan frame under the high-penetration imaging mode is higher than that under the conventional imaging mode.

18. The ultrasound imaging method according to claim 17, wherein selecting a current imaging mode from a conventional imaging mode and a high-penetration imaging mode, comprises:
obtaining a depth of a target region from an ultrasound image, and when the depth of the target region is greater than or equal to a target depth threshold, selecting the high-penetration imaging mode as the current imaging mode; wherein the target region comprises any one of: a region selected by a user in the ultrasound image, and a lesion region recognized based on the ultrasound image;
calculating a signal intensity obtained by scanning under the conventional imaging mode, and when the signal intensity is less than a signal intensity threshold, switching from the conventional imaging mode to the high-penetration imaging mode; and
calculating a signal-to-noise ratio of a signal obtained by scanning under the conventional imaging mode, and when the signal-to-noise ratio is less than a ratio threshold, switching from the conventional imaging mode to the high-penetration imaging mode.

19. The ultrasound imaging method according to claim 18, wherein the second scanning parameter is configured such that the ultrasound imaging system meets a safety index when working; the safety index includes at least one of: a mechanical index MI, a temperature, and a thermal index TI.

20. The ultrasound imaging method according to claim 19, wherein an display characteristic of a ultrasound image under the high-penetration imaging mode is higher than a ultrasound image under the conventional imaging mode, and the display characteristic comprises at least one of an image penetration, an image clarity and a richness of image details.
